(19) United States
(12) Patent Application Publication
(10) Pub. No.: US 2024/0238180 A1
(43) Pub. Date: Jul. 18, 2024

(54) SELF-CURING ACRYLIC FORMULATIONS CONTAINING N-ACETYL CYSTEINE FOR HAIR STRAIGHTENING

(71) Applicant: EASYFIX HAIR DESIGN LTD., Tel Aviv (IL)

(72) Inventors: Moshe UZAN, Petah Tikva (IL); Steve DAREN, Nes Ziona (IL); Ehud GAZIT, Ramat Hasharon (IL)

(21) Appl. No.: 18/553,878

(22) PCT Filed: Apr. 4, 2022
(86) PCT No.: PCT/IL2022/050353

§ 371 Date: Oct. 4, 2023

Related U.S. Application Data
(60) Provisional application No. 63/170,596, filed on Apr. 5, 2021.

(51) Int. Cl. A61K 8/44; A45D 7/06; A61K 8/81; A61K 8/86; A61Q 5/04

(52) U.S. Cl. CPC A61K 8/447; A45D 7/06; A61K 8/8152; A61K 8/86; A61Q 5/04; A61K 2800/5422

(57) ABSTRACT

The present invention provides hair straightening ready-to-use formulation comprising acrylic polymers configured to crosslink upon drying and thereby to form a film that is able to preserve hair in straightened conformation and an organic compound comprising at least one thiol group. The cosmetic formulations of the present invention are ready-to-use formulations and may be applied frequently.

### FIELD OF THE INVENTION

The present invention relates to hair styling formulations comprising an acrylic emulsion comprising acrylic-based polymers that undergo crosslinking upon drying together with an N-acetylated thiol-containing amino acid. The present invention further provides the hair-dressing compositions comprising a cosmetically acceptable aqueous medium and a hair treatment process using same.

### BACKGROUND OF THE INVENTION

Women of all ages often prefer to style their hair long and straight. The majority of women, however, do not have such hair but rather wavy, curly or dispersed one. Different hair styling procedures are known to straighten curly hair. Previously known processes used to straighten curly hair are both expensive and damaging to the hair fibers and therefore may not be used more than at intervals of several months or more. Known procedures of hair straightening involve changing the hair chemistry by attacking the sulfur-sulfur bonds in keratin, the main molecule in hair.

With a keratin treatment commonly known as the Brazilian Technique, the protein keratin formula is infused with an iron into freshly washed hair using intense heat, thus only impacting the outside layer of the hair. This procedure involves reacting the keratin with the toxic chemical formaldehyde. This procedure is banned in many countries. The second procedure, known as the Japanese procedure or thermal reconditioning, involves a chemical treatment and then heating the hair to a high temperature with an iron. Japanese hair straightening works by changing the inner bonds of hair. This causes the keratin to start to decompose and the hair can be straightened. This procedure is expensive, time-consuming, and may cause long term damage to the hair.

Use of acrylic polymers, also referred to as acrylates, is well known in many fields and industries such as the paint and coating industry, cosmetics, medicine and agriculture. This use is further exemplified by U.S. Pat. No. 4,217,396.

The paints and coatings industry relies heavily on water-based acrylic emulsions. To aid in the formation of water and solvent-resistant solid coatings, self-cross-linking polymeric dispersants have been developed. In such systems, all of the reactants for cross-linking are present in the emulsion and may be stable for many years. The cross-linking may be triggered by the evaporation of the water, change of pH or curing at elevated temperatures. Photo-curing is also an option. The incorporation of crosslinking chemistry in waterborne coatings or film forming compositions is recognized to provide a particularly effective means of enhancing the mechanical strength, chemical stability and solvent resistance of the final film. This process of coating formation is described among others by Buckmann et al. (European Coatings Journal, 2001, 6, 53-60). Film-forming acrylic polymers have even been used to increase storage stability of cut flowers as described in U.S. Pat. No. 8,664,161.

In the cosmetics and personal-care industries, many formulations of shampoos, creams and conditioners utilize water-based acrylic emulsions. These are considered to be safe and reliable. Acrylate copolymers such as Luvimer® (BASF), Balance@ (Akzo Nobel) or Acudyne® (Rohm and Haas) are used in the hair care industry. Use of emulsions of acrylic polymers for hair styling is described for example in U.S. Pat. Nos. 9,249,247, 9,345,656 and 9,717,673. U.S. Pat. No. 9,365,739 describes waterborne two component polyurethane coating compositions comprising alicyclic rigid polyols further comprising a vinyl or acrylic emulsion polymer. WO 2013/103528 describes polymers containing an acetoacetate moiety.

WO 2019234733 to the present inventors describes hair straightening composition comprising acrylic polymers configured to crosslink upon drying and thereby to form a film that is able to preserve hair in straightened conformation. Also provided are hair straightening cosmetic formulations.

Cruz et al., RSC Advances 2017,7,51581-51592 describe a mild chemical treatment to rearrange the disulfide bonds in keratin, the main ingredient of hair. They reported that three cysteine-containing peptides called PepE, PepG and KP showed the highest ability to interact with a keratin peptide molecule and to improve hair mechanical properties and straightening efficiency. However, peptide sequences are relatively expensive and not readily available on a commercial scale.

Cysteines were previously suggested as being useful in cold-hair waving arts. However, as disclosed e.g., in U.S. Pat. No. 4,992,267, cysteine, used as a main hair-straightening agent has certain disadvantages as relatively high amounts of upwards of about 3 weight percent to about 20 weight percent of cysteine (or a cysteine derivative) are reportedly required to effect a beneficial change in the configuration of the hair.

There exists a need in a market for a user-friendly formulation applied to curly or wavy hair, preferably at home but also in a hair salon, that can be applied at frequent intervals for temporary straightening of the hair. The formulation should be applied at moderate temperatures and without the use of toxic chemicals that alter the hair structure chemically.

### SUMMARY OF THE INVENTION

The present invention provides hair styling, and specifically hair straightening, ready-to-use formulations that have a dual mode of action; the formulations do not impair the hair being treated while avoiding the use of excessive heating and harsh chemical reactions. It is now disclosed for the first time that the addition of N-acetyl cysteine (NAC) to hair straightening formulations comprising self-cross-linking acrylic polymers significantly and even synergistically improved the hair straightening effects of the latter. Such formulations provided enhanced maintenance of the wet shape of the hair even after the hair has been dried as exemplified hereinbelow. Hair obtained after treatment with the formulation of the present invention has an extended look, lower volume and is smoother to the touch than when either of the materials is used individually. It is now disclosed that the combination according to the present invention achieves a prolonged effect and favorable condition of the treated hair compared to the previously known treatments.

The combined use of the thiol-containing amino acids and the acrylic polymers is also advantageous as it permits the use of sufficiently low concentrations of the components and in particular of thiol-containing amino acids. Typically, a high concentration of such amino acids leads to an unpleasant smell, as exemplified hereinbelow. Thus, formulations comprising low concentrations of thiol-containing amino acids (up to 1 wt %) as disclosed in the present application are highly beneficial. At such low concentrations, thiol-containing amino acids such as N-acetyl cysteine have very little if any effect on hair. Despite the low concentration of thiol-containing amino acids, the formulations of the present invention are shown to be very efficient, even on very curly and frizzy hair types (types 3 and 4). It was previously seen that for such types of hair, the physical support of the cross-linking emulsion was not sufficient to fully straighten and could at best be rendered wavy. In addition, it was possible to maintain the pH of the formulations of the present invention at about 5.5, which is optimal for formulations applied on the scalp. Addition of the NAC also provides enhanced stability of the formulation.

It is further disclosed that multiple applications of the formulation improve the outcome. Personal care products containing a blend of acrylic polymers and NAC for the temporary or semi-permanent styling, revitalization, and in particular straightening of human hair, by applying to the hair fibers the formulations disclosed herein are provided. On the hair surface, the polymers are converted to a cross-linked film that is stable for hours or days which maintains styling and protects the hair against humidity. The hair is surrounded by a protective envelope and is consequently protected from external aggression and moisture. The envelope is able to preserve the styling even under harsh conditions such as high humidity. The formulations are suitable for use on mammalian hair, including but not limited to human hair, equine manes and tails and pet coats. Advantageously, the formulations of the present invention achieve the straightening effect without requiring any extreme chemical reaction or causing damage to the hair.

Without being bound to any particular theory, it is assumed that the coating is obtained by the formation of a film around hairs upon drying. Such a film-forming process can be described as proceeding in three stages:
Stage 1: As the water evaporates, the polymer emulsion particles come into close contact and start to agglomerate;
Stage 2: Particle deformation and packing; and
Stage 3: Polymer coalescence—chains entangle and react (cross-link with other polymers) and particle boundaries disappear. At this point, an essentially continuous film is formed.

The coating film around hair, formed by the polymers of the formulations of the present invention, provides a much better and longer-lasting hair styling than any known hair styling product utilizing acrylic polymers. The formation of the enhanced film is conferred by cross-linkers present within the polymers. The cross-linkers covalently bind polymers to form a polymeric net. The resulting film allows maintaining hair e.g., in straightened conformation, for a prolonged period even under humid conditions. NAC significantly and even synergically improves this effect.

In one aspect, the present invention provides a hair straightening formulation comprising from about 1 wt % to about 20 wt % of at least one acrylic-based polymer comprising acrylic units that act as cross-linkers that will form bonds between the polymer chains wherein the amount of said cross-linker units is from about 1% to about 25 wt % of said acrylic-based polymer, from about 0.05 wt % to about 6 wt % of a non-ionic surfactant, from 0.01 to 3 wt % of an organic compound comprising one or more thiol group, and a cosmetically acceptable aqueous carrier, wherein the polymers undergo cross-linking and thereby maintain the hair in a straightened configuration upon drying. According to some embodiments, the organic compound comprising one or more thiol groups has a molecular weight of from 50 to 1000 Da. According to some embodiments, the non-ionic surfactant is selected from a pharmaceutical or cosmetic grade surfactant. According to some embodiments, the formulation comprises from 0.1 to 1 wt % of the organic compound comprising one or more thiol groups. According to some embodiments, the formulation comprises up to 1 wt % of the organic compound comprising one or more thiol groups. According to some embodiments, the formulation comprises less than 1 wt % of the organic compound comprising one or more thiol groups. According to some embodiments, the organic compound comprising one or more thiol groups has a molecular weight of from 100 to 400 Da. According to some embodiments, the organic compound comprising one or more thiol groups is selected from an amino acid or derivative thereof, captopril, and glutathione. According to some embodiments, the organic compound comprising one or more thiol groups is selected from cysteine and derivative of cysteine. According to some embodiments, the derivative of cysteine is selected from N-acetyl cysteine, cysteine methyl ester, acetylcysteine, carbocysteine, fudosteine and erdosteine. According to some embodiments, the organic compound comprising one or more thiol group is N-acetyl cysteine.

According to some embodiments, the polymer is derived from (meth)acrylic monomers and/or (meth)acrylic oligomers such as monoacrylate oligomer, isobornyl acrylate, butyl acrylate, lauryl methacrylate, and any combination thereof. According to one embodiment, the polymer is derived from a combination of (meth)acrylic monomers and acrylate containing oligomers. According to one embodiment, the polymer is derived from a combination of monoacrylate oligomer and isobornyl acrylate. According to another embodiment, the polymer is derived from a combination of monoacrylate oligomer, isobornyl acrylate and butyl acrylate.

According to one embodiment, the polymer is derived from a combination of monoacrylate oligomer, isobornyl acrylate and lauryl methacrylate. In other words, the polymer comprises the units (the moieties) of the monomers from which it is derived. According to some embodiments, the monoacrylate oligomer is epoxy monoacrylate oligomer. According to some embodiments, the cross-linker unit is 2-(methacryloyloxy)ethylacetoacetate (AAEM). According to some embodiments, the non-ionic surfactant is selected from the group consisting of ethoxylated and/or propoxylated alcohol or ester, tyloxapol, polysorbate (TWEEN), PPG-5-ceteth-20, PEG-20 sorbitan isostearate, alkoxylated alcohols, alkoxylated polyol esters, decyl glucoside, coco-glucoside, sucrose laurate, sorbitol, polyoxyethylene, sorbitan monolaurate, Laureth comprising from 20 to 40 units of ethylene oxide such as Laureth-20, Laureth-21, Laureth-25, Laureth-30, Laureth-38, Laureth-40, Disponil AFX 4060 (modified ethoxylated fatty alcohols polyoxyethylene 40) and Sabowax SE40 (polyethylene glycol-40 stearate) and any combination hereof. According to some embodiments, the hair straightening formulation of the present invention comprises from about 0.2 wt % to about 4 wt % or from about 0.2 wt % to about 1.5 wt % of said surfactant. According to some embodiments, the ready-to-use hair straightening formulation of the present invention comprises from about 0.2 wt % to about 2 wt %, from 0.3 to 3, from 0.2 to 3 wt %, from 0.4 to 1.5 or from 0.2 to 1.2 wt % of the surfactant or combination thereof.

According to a particular embodiment, the hair straightening ready-to-use formulation of the present invention comprises from about 1 wt % to about 10 wt % of said polymer, a cross-linker in the amount of about 10 wt % to about 20 wt % of said polymer, from 0.05 to 0.5 wt % of the organic compound comprising one or more thiol group and from about 0.5 wt % to about 2 wt % of said non-ionic surfactant in an aqueous carrier. According to some embodiments, the glass transition temperature (Tg) of the polymer is from about 10° C. to about 50° C. According to other embodiments, the polymers of the formulation of the present invention undergo crosslinking upon drying. According to some embodiments, the pH of the formulation is from 5 to about 7.

According to some embodiments, the formulation of the present invention further comprises cosmetically acceptable ingredients selected from the group consisting of preservatives, thickeners, viscosity modifiers, fatty alcohols, pH adjusting agents, buffers, coloring agents, guar gums, perfumes, PEGs, essential oils, silicone fluid dimethicone, hydroxypropyl cellulose, carboxymethyl cellulose and any combination thereof.

According to another embodiment, the present invention provides a method of straightening hair comprising applying the hair straightening formulation of the present invention to hair and drying the formulation. According to some embodiments, the method further comprises the step of straightening the hair by combing or brushing before or during drying. According to some embodiments, the formulation of the present invention is applied to dry or wet hair. According to another embodiment, the formulation is dried by gentle heat or at room temperature. According to some embodiments, the formulation of the present invention is applied a plurality of times.

### BRIEF DESCRIPTION OF DRAWINGS

FIG. 1 shows typical untreated swatches of Brazilian hair type 2.

FIG. 2 shows swatches of Brazilian hair type 2 after treatment with formulations 1-7 disclosed in Example 6. FIG. 2A shows swatches after the treatment, FIG. 2B—swatches after 3 days in humid conditions.

FIG. 3 shows the effect of one (FIG. 3A), two, (FIG. 3B), three (FIG. 3C) and four (FIG. 3D) applications of hair straightening formulations comprising 0.3 wt % of NAC.

FIG. 4 shows the effect of one (FIG. 4B) or three (FIG. 4C) applications of hair straightening formulations comprising different concentrations of NAC. FIG. 4A shows swatches before application.

FIG. 5 shows the effect different modes of drying. FIG. 5A—before application, FIG. 5B, FIG. 5C and FIG. 5D shows swatches after 1, 2 and 3 applications respectively. Swatch 1—DDW—air drying, Swatch 2—DDW+EF20%+NAC 0%+air drying; Swatch 3—DDW+EF20%+NAC 0.3%+air drying; Swatch 4—DDW+EF20%+NAC 0.3%+Blow dryer; Swatch 5—DDW+EF20%+NAC 0.3%+Flat Iron.

FIG. 6 shows the comparison between drying methods after hair straightening using the formulation of the present invention: air drying and flat iron.

### DETAILED DESCRIPTION OF THE INVENTION

The present invention relates to formulations for preserving straightened hair in an elongated form and conferring hair smoothness, promoting a lustrous shine and having a good mending effect. The formulation of the present invention allows the reduction of the total volume of curly and frizzy hair. The formulations of the present invention provide also protection against humidity and other ambient stresses. It was further seen that increasing the number of applications of the formulation improves its effect.

According to one aspect, the present invention provides a hair styling formulation from about 1 wt % to about 20 wt % of at least one acrylic-based polymer comprising acrylic units that act as cross-linkers that will form bonds between the polymer chains and from about 0.05 wt % to about 10 wt % of a non-ionic surfactant, wherein the amount of said cross-linker units is from about 1% to about 25 wt % of said acrylic-based polymer, from 0.01 to 3 wt % of an organic compound comprising one or more thiol group and having a molecular weight of from 50 to 1000 Da, and a cosmetically acceptable carrier, wherein the formulation undergoes cross-linking and thereby maintains the hair in the styled conformation upon drying. According to one embodiment, hair styling is hair straightening. Thus, according to the aspect, the present invention provides a hair straightening ready-to-use formulation comprising from about 1 wt % to about 20 wt % of at least one acrylic-based polymer comprising acrylic units that act as cross-linkers wherein the amount of said cross-linker units is from about 1% to about 25 wt % of said acrylic-based polymer, and from about 0.05 wt % to about 6 wt % of a non-ionic surfactant, from 0.01 to 3 wt % of an organic compound comprising one or more thiol group and having a molecular weight of from 50 to 1000 Da, and a cosmetically acceptable carrier, wherein the polymers undergo cross-linking and thereby straighten the hair upon drying. According to one embodiment, the cross-linkers form bonds between the polymer chains upon drying of the formulation. According to some embodiments, the formulation comprises up to 1 wt % of the compound comprising one or more thiol groups. Thus, according to some embodiments, the present invention provides a hair straightening ready-to-use formulation comprising from about 1 wt % to about 20 wt % of at least one acrylic-based polymer comprising acrylic units that act as cross-linkers wherein the amount of said cross-linker units is from about 1% to about 25 wt % of said acrylic-based polymer, and from about 0.05 wt % to about 6 wt % of a non-ionic surfactant, from 0.01 to 0.9 wt % of an organic compound comprising one or more thiol group and having a molecular weight of from 50 to 1000 Da, and a cosmetically acceptable carrier, wherein the polymers undergo cross-linking and thereby straighten the hair upon drying.

The term “hair straightening” encompasses also the meaning of preserving or maintaining the straightened hair straight, e.g., maintaining hair straightened after wetting the hair. Thus, the terms “hair straightening”, “preserving hair in straight conformation”, “preserving hair in straightened conformation”, “maintaining hair straight”, “maintaining the hair straightened” and “maintaining straightened hair straight” may be used herein interchangeably. According to one embodiment, the length of the straightened hair upon drying the formulation is up to 200%, up to 160% or up to 140% of the hair's length in un-straightened conformation.

According to another embodiment, the length of the straightened hair upon drying the formulation is from 101% to 130%, 105% to 125%, or from 110% to 120% of the hair's length in un-straightened conformation. According to another embodiment, the length of the straightened hair upon drying the formulation is from 101% to 130%, 105% to 125%, or from 110% to 120% of the hair's length in un-straightened conformation even in comparison to the composition of the present invention without the NAC. The un-straightened conformation means the conformation of dry and untreated hair. In one embodiment, the un-straightened conformation comprises curly, wavy or kinky hair. According to some embodiments, straight or straightened conformation does not necessarily mean that the hair is perfectly straight, but rather that the hair is longer and/or straighter than its initial state.

As used herein, the terms “emulsion”, “stock composition”, “stock emulsion” and “polymer emulsion” can be used interchangeably and refer to a stock emulsion from which a final product is prepared.

As used here, the terms “formulation” and “ready-to-use formulation” refer to the final hair care cosmetic product. According to some embodiments, the formulation is a cosmetic formulation. According to other embodiments, the formulation is ready-to-use hair care formulation, which may be used directly on hair.

As used herein, the terms “polymer” and “polymer chain” are used interchangeably and refer to a single polymeric chain that did not undergo crosslinking with another polymeric chain. According to the teaching of the present invention, polymer chains upon crosslinking form a network of polymers—a film.

According to some embodiments, the ready-to-use hair styling composition, and specifically hair straightening composition is stable for at least 2, 4, 6, 8, 10, 12, 14, 16, 18 or 24 months. According to some embodiments, the ready-to-use hair styling composition is stable for 1 year or more, for 2 years or more or for 3 years or more.

The carrier of the present invention is adapted to the topical mode of administration and therefore being a topically/cosmetically/dermatologically acceptable carrier. The terms “cosmetically acceptable carrier”, “dermatologically acceptable carrier” and “carrier for topical formulations” are used herein interchangeably and refer to a carrier, excipient or diluent that is suitable for use in contact with the skin without undue toxicity, incompatibility, instability, irritation, allergic response, and the like. It is appreciated that all carriers, excipients and diluents which can be added to the cosmetic formulation of the present invention are approved for human and animal for cosmetic use. According to some embodiments, the carrier is water, e.g., double distilled water.

According to some embodiments, the ready-to-use formulation of the present invention temporarily preserves hair in the styled conformation. According to some embodiments, the ready-to-use formulation of the present invention temporarily preserves hair in the straightened conformation. According to another embodiment, the formulation of the present invention preserves or maintains hair in straightened conformation from several hours to several days. According to one embodiment, the formulation of the present invention preserves hair in the straightened conformation for from 1 to 24, from 3 to 20, from 4 to 16, or from 6 to 12 hours, provided that that hair is not wetted, washed or shampooed. According to one embodiment, the formulation of the present invention preserves hair in the straightened conformation for 1 to 5, from 2 to 4, or for 3 days, provided that that hair is not wetted, washed or shampooed. According to other embodiments, the formulation of the present invention preserves hair in the straightened conformation for 3 to 14, for 5 to 12 or for 7 to 10 days.

According to one embodiment, the hair straightening formulation comprises from about 1 wt % to about 20 wt % of acrylic-based polymers. According to one embodiment, the hair straightening formulation comprises from about 1 wt % to about 15 wt % or about 2 to about 12 wt % of acrylic-based polymers. According to another embodiment, the formulation comprises from about 3 wt % to about 10 wt % of acrylic-based polymers. According to yet another embodiment, the formulation comprises from about 2 wt % to about 8 wt % of acrylic-based polymers. According to another embodiment, the formulation comprises from about 3 wt % to about 10 wt %, from about 2 to about 9 wt %, from about 3 wt % to about 8 wt %, from about 4 wt % to about 6 wt %, or from about 3 wt % to about 6 wt % of acrylic-based polymers.

According to another embodiment, the polymer comprises from about 2 wt % to about 25 wt % of acrylic-based cross-linker units. According to another embodiment, the polymer comprises from about 2.5 wt % to about 20 wt % of the cross-linker. According to some embodiments, the polymer comprises from about 2.5 to 18 wt %, from 3 to 17 wt %, from 3.5 to 16 wt %, from 4 to 15 wt % from 4.5 to 14 wt %, from 5 to 12 or from 7 wt % to about 18 wt % of the cross-linkers. According to some embodiments, the polymer comprises from about 10 wt % to about 20 wt % of the cross-linker units. According to certain embodiments, the amount of the cross-linker units is from about 6% to about 20 wt % of said acrylic-based polymer. According to one embodiment, the amount of the cross-linker units is from about 8% to about 18 wt % of said acrylic-based polymer. According to some embodiments, the amount of the cross-linker units is from about 12% to about 18 wt % of said acrylic-based polymer. According to some embodiments, the amount of the cross-linker units is from about 2% to about 10 wt % of said acrylic-based polymer. According to one embodiment, the amount of the cross-linker units is from about 3 to about 9 wt % of said acrylic-based polymer. According to another embodiment, the amount of said cross-linker units is from about 4% to about 8 wt % of said acrylic-based polymer. According to some embodiments, the amount of the cross-linker units is about 6, about 8, about 10, about 12, about 13, about 14, about 15, about 16, about 17, about 18, about 19 or about 20 wt % of said acrylic-based polymer. According to some embodiments, the formulation comprises from 0.1 to 2 wt % of the cross-linker units. According to other embodiments, the formulation comprises from 0.2 to 1.5 wt % of the cross-linkers. According to one embodiment, the formulation comprises from 0.3 to 1.3 wt % of the cross-linkers. According to another embodiment, the formulation comprises from 0.4 to 1.2 wt % of the cross-linkers.

According to some embodiments, the formulation comprises from about 2 wt % to about 15 wt %, from about 2 to about 12 wt %, from about 3 wt % to about 10 wt %, or from about 3 wt % to about 8 wt % of acrylic-based polymers and the polymer comprises from about 5 wt % to about 25 wt % of the cross-linker or from about 10 wt % to about 20 wt % of the cross-linker.

According to any one of the above embodiments, the formulation comprises from about 0.05 to about 6 wt % or from 0.1 wt % to about 7 wt % of non-ionic surfactant. According to certain embodiments, the formulation comprises from about 0.2 to about 6 wt % of non-ionic surfactant. According to one embodiment, the formulation comprises from about 0.3 wt % to about 5.5 wt %, from about 0.5 wt % to about 5 wt %, from about 0.75 to about 4.5 wt % of the non-ionic surfactant. According to one embodiment, the formulation comprises from about 0.4 wt % to about 6 wt % of a non-ionic surfactant. According to another embodiment, the formulation comprises from about 0.4 wt % to about 2 wt % of a non-ionic surfactant. According to yet another embodiment, the formulation comprises from about 0.6 wt % to about 1.5 wt % of a non-ionic surfactant.

According to some embodiments, the formulation of the present invention comprises from 0.01 to 3 wt % of an organic compound comprising one or more thiol group and having molecular weight of from 50 to 1000 Da. According to some embodiments, the formulation comprises from 0.05 to 2.5 wt %, from 0.07 to 2.3 wt %, from 0.1 to 2 wt %, from 0.15 to 1.5 wt %, from 0.2 to 1 wt %, or from 0.25 to 0.8 wt % of the organic compound comprising one or more thiol group. According to some embodiments, the formulation comprises from 0.05 to 1.2 wt %, from 0.07 to 1 wt %, from 0.09 to 0.9 wt %, from 0.1 to 0.8 wt %, from 0.15 to 0.75 wt %, from 0.2 to 0.7 wt %, from 0.25 to 0.6 wt % of the organic compound comprising one or more thiol groups. According to some embodiments, the formulation comprises from 0.05 to 0.9 wt % of the organic compound comprising one or more thiol groups. According to some embodiments, the formulation comprises from 0.1 to 0.8 wt % of the organic compound comprising one or more thiol groups. According to some embodiments, the formulation comprises less than 1 wt % of the organic compound comprising one or more thiol groups. According to some embodiments, the formulation comprises up to 1 wt % of the organic compound comprising one or more thiol groups. The terms “less than” and “no more than” are used herein interchangeably and refer to a value that is less than the named value but more than zero. Similarly, the term “up to” does not include the upper value.

According to some embodiments, the organic compound comprising one or more thiol group has a molecular weight of from 50 to 600 Da or from 100 to 400 Da.

According to some embodiments, the organic compound comprising one or more thiol group is selected from a thiol comprising amino acid or derivative thereof. According to some embodiments, the organic compound comprising one or more thiol group is cysteine. According to another embodiment, the organic compound comprising one or more thiol group is a derivative of cysteine. According to one embodiment, the organic compound comprising one or more thiol group is selected from N-acetyl cysteine, cysteine methyl ester, acetylcysteine, carbocysteine, fudosteine and erdosteine. According to one embodiment, the organic compound comprising one or more thiol group is N-acetyl cysteine. According to some embodiments, the formulation of the present invention comprises from 0.05 to 2.5 wt %, from 0.07 to 2.3 wt %, from 0.1 to 2 wt %, from 0.15 to 1.5 wt %, from 0.2 to 1 wt %, or from 0.25 to 0.8 wt % of N-acetyl cysteine. According to some embodiments, the formulation comprises from 0.05 to 1.2 wt %, from 0.07 to 1 wt %, from 0.09 to 0.9 wt %, from 0.1 to 0.8 wt %, from 0.15 to 0.75 wt %, from 0.2 to 0.7 wt %, from 0.25 to 0.6 wt % of N-acetyl cysteine. According to some embodiments, the formulation comprises from 0.1 to 0.6 wt % of N-acetyl cysteine. According to some embodiments, the formulation comprises from 0.1 to 0.4 wt % of N-acetyl cysteine. According to some embodiments, the formulation comprises from 0.2 to 0.4 wt % of N-acetyl cysteine. According to some embodiments, the formulation comprises about 0.3 wt % of N-acetyl cysteine. According to some embodiments, the formulation comprises from 0.05 to 0.9 wt % of NAC. According to some embodiments, the formulation comprises from 0.1 to 0.8 wt % of NAC. According to some embodiments, the formulation comprises less than 1 wt % of NAC. According to some embodiments, the formulation comprises up to 1 wt % of NAC.

According to some embodiments, the formulation comprises from about 2 wt % to about 15 wt %, from about 2 wt % to about 12 wt %, from about 3 wt % to about 10 wt %, or from about 2 wt % to about 8 wt % of acrylic-based polymers, from about 0.2 wt % to about 4 wt % non-ionic surfactant and the polymer comprises from about 5 wt % to about 20 wt % of the cross-linker or from about 7 wt % to about 18 wt %, or from about 12 to about 18 wt % of the cross-linker units.

According to one embodiment, the present invention provides a hair straightening ready-to-use formulation from about 1 wt % to about 20 wt % of at least one acrylic-based polymer comprising acrylic-based cross-linker units and from about 0.05 wt % to about 4.5 wt % of a non-ionic surfactant, wherein the amount of said cross-linkers is from about 2.5 to about 25 wt % of said acrylic-based polymer and the non-ionic surfactant is selected from a pharmaceutical or cosmetic grade surfactant, from 0.01 to 0.9 wt % of an organic compound comprising one or more thiol group and having molecular weight of from 50 to 1000 Da, and a cosmetically acceptable carrier, wherein the formulation straightens the hair upon drying. According to another embodiment, the formulation comprises from about 2 wt % to about 10 wt % of said polymer comprising cross-linker units in the amount of about 5 wt % to about 20 wt % of said polymer, and from about 0.3 wt % to about 3 wt % of said non-ionic surfactant.

According to some embodiments, the formulation of the present invention comprises from about 1.5 wt % to about 12 wt % of said polymer comprising cross-linkers in the amount of about 3 wt % to about 20 wt % of said polymer, and from about 0.2 wt % to about 4 wt % of said non-ionic surfactant, and from 0.2 to 0.8 N-acetyl cysteine. According to some embodiments, the formulation of the present invention comprises from about 5 wt % to about 12 wt % of the polymer comprising cross-linkers in the amount of about 3 wt % to about 20 wt % of said polymer, from about 0.2 wt % to about 4 wt % of said non-ionic surfactant, and from 0.2 to 0.8 of N-acetyl cysteine. According to some embodiments, the formulation of the present invention comprises from about 6 wt % to about 10 wt % of the polymer comprising cross-linkers in the amount of about 3 wt % to about 20 wt % of said polymer, from about 0.2 wt % to about 4 wt % of said non-ionic surfactant, and from 0.2 to 0.6 of N-acetyl cysteine.

According to some of the above embodiments, the acrylic-based polymer is derived from acrylic monomers. According to other of the above embodiments, the acrylic-based polymer is derived from methacrylic monomers. According to other embodiments, the acrylic-based polymer is derived from acrylic oligomers. According to certain embodiments, the acrylic-based polymer is derived from a mixture of (meth)acrylic monomers and acrylic oligomers such as mono-acrylic oligomers. According to some embodiments, the acrylic-based polymer is derived from a mixture of acrylic monomers such as acrylic monomer, methacrylic monomers and a combination thereof, and mono-acrylic oligomers. According to some embodiments, the mono-acrylic oligomers is epoxy monoacrylate oligomer. The term “acrylic-based polymer”, as used herein has the meaning that at least 50 wt % of the monomers moieties forming said polymer are acrylic moieties. The term “derived” has the meaning of obtained from said monomers via a chemical reaction, i.e., via polymerization of said monomers. It is contemplated by the teaching of the present invention that the polymer may contain non-acrylic monomer moieties. According to any one of the above embodiments, the content of acrylic units that form the polymer is from 85 to 100 wt % of said polymer. Thus, according to one embodiment, the polymer comprises from 85 to 100 wt %, from 90 to 98 wt %, from 95 to 97 wt % or from 95 to 99 wt % of acrylic moieties. According to one embodiment, the cross-linker unit and acrylic monomer and/or acrylic oligomers moieties collectively constitute from 85 to 100% of the polymer. According to another embodiment, the cross-linker unit and acrylic monomer and/or acrylic oligomers moieties collectively constitute from 90 to 99 wt % from 90 to 98 wt %, 95 to 97 wt % or from 95 to 99 wt % of the polymer. According to some embodiments, the polymer consists essentially of acrylic based units and acrylic-based cross linker units. According to other embodiments, the polymer consists of acrylic moieties and acrylic-based cross-linker moieties.

The terms “acrylic monomers” and “acrylate monomers” are used herein interchangeably and refer to monomers based on the structure of acrylic acid and/or methacrylic acid. The term “acrylic monomers” also encompasses methacrylate monomer. The term “acrylic oligomer” as used herein refers to oligomers comprising an acrylic or methacrylic group. According to one embodiment, the acrylic monomers are selected from the group consisting of isobornyl acrylate, butyl acrylate, lauryl methacrylate, and any combination thereof. According to one embodiment, the acrylic monomer is any alkyl acrylate. The term “alkyl” as used herein typically means a straight or branched saturated hydrocarbon radicals having 1-18 carbon atoms and includes, e.g., methyl, ethyl, n-propyl, isopropyl, n-butyl, sec-butyl, isobutyl, tert-butyl, n-pentyl, 2,2-dimethylpropyl, and the like. According to other embodiments, the acrylic monomer is selected from methacrylate, isobornyl methacrylate, butyl methacrylate, and lauryl methacrylate.

According to one embodiment, the acrylic monomer is any alkyl methacrylate. According to some embodiments, acrylic oligomer is a monoacrylate oligomer. According to some embodiments, acrylic oligomer is epoxy monoacrylate oligomer. As such, the term oligomer refers to the side chains of the monoacrylate. According to one embodiment, the monoacrylate oligomer is 2-hydroxy-3-(prop-2-enoyloxy) propyl 2-methyl-2-propylhexanoate (CN152 of Sartomer having CAS number 444649-70-1). According to another embodiment, the monoacrylate oligomer is selected from polyester mono-(meth)acrylate, urethane mono-(meth)acrylate and amine-modified mono-(meth)acrylate.

According to one embodiment, the polymer is derived from a combination of monoacrylate oligomer and isobornyl acrylate. According to one embodiment, the weight ratio between monoacrylate oligomer and isobornyl acrylate is from 1:10 to 10:1, from 1:9 to 9:1, from 1:8 to 8:1, from 1:7 to 7:1, from 1:6 to 6:1, from 1:5 to 5:1, from 1:4 to 4:1, from 1:3 to 3:1 or from 1:2 to 2:1. According to certain embodiments, the weight ratio between monoacrylate oligomer and isobornyl acrylate is 1.5:1 to 1:1.5. According to one embodiment, the monoacrylate oligomer is 2-hydroxy-3-(prop-2-enoyloxy)propyl 2-methyl-2-propylhexanoate. According to some embodiments, acrylic oligomer is epoxy monoacrylate oligomer.

According to another embodiment, the polymer is derived from a combination of monoacrylate oligomer, isobornyl acrylate and butyl acrylate. According to one embodiment, the weight ratio between monoacrylate oligomer, isobornyl acrylate and butyl acrylate is from 10:10:1 to 1:1:10 or about from 10:9:2. According to another embodiment, the weight ratio between monoacrylate oligomer, isobornyl acrylate and butyl acrylate is from 10:10:1 to 1:10:1. According to a further embodiment, the weight ratio between monoacrylate oligomer, isobornyl acrylate and butyl acrylate is from 10:10:1 to 10:1:1. According to a certain embodiment, the weight ratio between monoacrylate oligomer, isobornyl acrylate and butyl acrylate is from 10:10:1 to 10:10:10. According to one embodiment, the monoacrylate oligomer is 2-hydroxy-3-(prop-2-enoyloxy)propyl 2-methyl-2-propylhexanoate. According to some embodiments, acrylic oligomer is epoxy monoacrylate oligomer.

According to yet another embodiment, the polymer is derived from a combination of monoacrylate oligomer, isobornyl acrylate and lauryl methacrylate. According to one embodiment, the weight ratio between monoacrylate oligomer, isobornyl acrylate and lauryl methacrylate is from 10:10:1 to 1:1:10 or about from 10:9:2. According to another embodiment, the weight ratio between monoacrylate oligomer, isobornyl acrylate and lauryl methacrylate is from 10:10:1 to 10:1:1. According to one embodiment, the weight ratio between monoacrylate oligomer, isobornyl acrylate and lauryl methacrylate is from 10:10:1 to 1:10:1. According to a certain embodiment, the weight ratio between monoacrylate oligomer, isobornyl acrylate and lauryl methacrylate is from 10:10:1 to 10:10:10. According to one embodiment, the monoacrylate oligomer is 2-hydroxy-3-(prop-2-enoyloxy)propyl 2-methyl-2-propylhexanoate. According to some embodiments, acrylic oligomer is epoxy monoacrylate oligomer.

According to any one of the above embodiments, the polymer comprises the units (the moieties) of the monomers from which it is derived. According to some embodiments, the ratios of the monomer units within the polymer are present at the ratios as defined hereinabove.

According to any one of the above embodiments, the cross-linker is 2-(methacryloyloxy)ethylacetoacetate (AAEM) or a derivative thereof. According to another embodiment, the cross-linker is selected from N-methylol acrylamide, methacrylic acid derivative, aziridines with acid groups; amines with epoxy(oxirane); alkoxy silanes; n-methylol acrylamide; and diacetone acrylamide dihydrazide. The terms “cross-linker”, “cross-linker unit”, “acrylic cross-linker” and “acrylic cross-linker unit” are used herein interchangeably and include both bifunctional and multifunctional units. According to the teaching of the present invention, cross-linkers are parts of a polymer chain and are capable of binding (cross-linking) to another polymer chain thereby producing a net or a film of polymers. According to one embodiment, the polymer upon drying and crosslinking forms a film around hair. Thus according to any one of the embodiments, the present invention provides a hair straightening formulation comprising from about 1 wt % to about 20% of at least one acrylic-based polymer comprising acrylic units that act as cross-linkers capable of forming bonds between the polymers and from about 0.05 wt % to about 6 wt % of a non-ionic surfactant, from 0.01 to 3 wt % of an organic compound comprising one or more thiol group wherein the amount of said cross-linker units is from about 2.5% to about 25 wt % of said acrylic-based polymer and the non-ionic surfactant is selected from a pharmaceutical or cosmetic grade surfactant, and wherein the polymer chains undergo cross linking and thereby straightens the hair upon drying. According to some embodiments, the formulation maintains the straightened hair in straight conformation.

According to any one of the above embodiments, the formulation comprises a non-ionic surfactant. According to some embodiments, the non-ionic surfactant is selected from a pharmaceutical or cosmetic grade surfactant. According to one embodiment, the non-ionic surfactant is selected from the group consisting of ethoxylated and/or propoxylated alcohol or ester, polyoxyether, Disponil AFX 4060 (fatty acid alcohol polyglycol ether or more specifically modified ethoxylated fatty alcohols polyoxyethylene (POE) 40), Sabowax SE40 (polyethylene glycol-40 stearate, CAS number 9004-99-3), tyloxapol, polysorbate (TWEEN), PPG-5-ceteth-20, PEG-20 sorbitan isostearate, alkoxylated alcohols, alkoxylated polyol esters, decyl glucoside, coco-glucoside, sucrose laurate, sorbitol, polyoxyethylene, sorbitan monolaurate and any combination hereof.

According to some embodiments, the non-ionic surfactant is a polyoxyether of lauryl alcohol. According to one embodiment, the polyoxyether of lauryl alcohol is selected from the group consisting of Laureth-1, Laureth-2, Laureth-3, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-21, Laureth-25, Laureth-30, Laureth-38, Laureth-40 and Laureth-50. The number in the name indicates the average number of units of ethylene oxide in the molecule. According to one embodiment, the non-ionic surfactant is selected from Laureth-16, Laureth-20, Laureth-21, Laureth-25, Laureth-30, Laureth-38, and Laureth-40. According to one embodiment, the non-ionic surfactant is Laureth-40. According to some embodiments, the surfactant is Laureth comprising from 20 to 60 units of ethylene oxide. According to other embodiments, the surfactant is Laureth comprising from 30 to 50 units of ethylene oxide. According to some embodiments, the formulation comprises from 0.1 to 3 wt % of Laureth having from 20 to 60 units, such as Laureth 40. According to one embodiment, the formulation comprises from 0.2 to 2.5 wt %, from 0.3 to 2 or from 0.3 to 1.5 wt % of Laureth surfactant, such as Laureth-40. According to some embodiments, the formulation comprises from 0.2 to 2.5 wt %, from 0.3 to 2 or from 0.3 to 1.5 wt % of Disponil 4060 AFX. According to some embodiments, the formulation comprises from 0.1 to 3 wt % of Sabowax SE40. According to one embodiment, the formulation comprises from 0.2 to 2.5 wt %, from 0.3 to 2 or from 0.3 to 1.5 wt % of Sabowax SE40. According to some embodiments, the formulation comprises from 0.5 wt % to 1.5 wt % or from 0.6 to 1.2 wt % of Sabowax SE40.

Nonionic surfactants, can be broadly defined as compounds produced by the condensation of alkylene oxide groups (hydrophilic in nature) with an organic hydrophobic compound, which may be aliphatic or alkyl aromatic in nature. Generally, the surfactant is a non-ionic alkylene oxide condensate of an organic compound that contains one or more hydroxyl groups. For example, ethoxylated and/or propoxylated alcohol or ester compounds or mixtures thereof are commonly available and are well known to those skilled in the art. Suitable surfactants include, but are not limited to, tyloxapol; POLOXAMER 4070; POLOXAMER 188; POLYOXYL 40 Stearate; POLYSORBATE 80, and POLYSORBATE 20, as well as various compounds sold under the trade name TWEEN (ICI American Inc., Wilmington, Del. U.S.A.), PLURONIC F-68 (trade name of BASF, Ludwigshafen, Germany for a copolymer of polyoxyethylene and polyoxypropylene).

According to any one of the above embodiments, the surfactant is a pharmaceutical or cosmetic grade surfactant. The terms “pharmaceutical grade” and “pharmaceutically acceptable” are used herein interchangeably and refer to molecular entities such as surfactants and formulations that do not produce adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards or the European medicines agency (EMA). According to some embodiments, the surfactant is a surfactant approved by FDA for human use. The terms “cosmetic grade” and “cosmetically acceptable” are used herein interchangeably and refer to any ingredient, such as surfactant, that is suitable for use in contact with the skin or hair of humans and lower animals without undue toxicity, incompatibility, instability, irritation, allergic response, and the like.

According to some embodiments, any other surfactant that can be applied on the skin and hair for a long period of time, may be used instead of or in addition to non-ionic surfactant. According to some embodiments, the surfactant that may be used instead of or in addition to non-ionic surfactant is a cationic, anionic, amphoteric and zwitterionic surfactant.

Non-limiting examples of cationic surfactants include quaternary ammonium salts, e.g., tetramethylammonium halides, alkyltrimethylammonium halides in which the alkyl group has from about 8 to 22 carbon atoms, for example octyltrimethylammonium chloride, dodecyltrimethylammonium chloride, hexadecyltrimethylammonium chloride, cetyltrimethylammonium chloride, and behenyltrimethylammonium chloride, benzyltrimethylammonium chloride, octyldimethylbenzyl-ammonium chloride, decetyldimethylbenzylammonium chloride, stearyldimethylbenzylammonium chloride, distearyldimethylammonium chloride, didodecyldimethylammonium chloride, dioctadecyldimethylammonium chloride, tallow trimethylammonium chloride, cocotrimethylammonium chloride, cetylpyridinium chloride and the other corresponding halides and hydroxides

Amphoteric and zwitterionic surfactants suitable for use in formulations of the invention may include alkyl amine oxides, alkyl betaines, alkyl amidopropyl betaines, alkyl sulfobetaines, alkyl glycinates, alkyl carboxyglycinates, alkyl amphopropionates, alkulamphoglycinates, alkyl amidopropyl hydroxy-sultaines, acyl taurates and acyl glutamates, wherein the alkyl and acyl groups have from about 8 to 19 carbon atoms. Examples include lauryl amine oxide, cocodimethyl sulphopropyl betaine and preferably lauryl betaine, cocamidopropyl betaine and sodium cocamphoproprionate.

Suitable anionic surfactants are the alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 8 to 18 carbon atoms and may be unsaturated. The alkyl ether sulfates, alkyl ether phosphates and alkyl ether carboxylates may contain from one to 10 ethylene oxide or propylene oxide units per molecule.

According to other embodiments, the formulation further comprises additional surfactants such as sodium dodecyl sulfate or alkyl benzene sulfonates. According to some embodiments, the alkyl benzene sulfonate is selected from branched alkyl benzene sulfonate and linear alkyl benzene sulfonate. According to another embodiment, linear alkyl benzene sulfonate is sodium dodecylbenzene sulfonate. According to one embodiment, the formulation comprises sodium dodecylbenzene sulfonate. According to some embodiments, the formulation comprises from 0.01 wt % to about 6 wt of sodium dodecylbenzene sulfonate. According to one embodiment, the formulation comprises from about 0.01 wt % to about 3 wt % or from 0.05 wt % to about 2 wt % or from 0.1 wt % to about 1.5 wt % of sodium dodecylbenzene sulfonate. According to another embodiment, the formulation comprises from about 0.08 wt % to about 0.3 wt % or from about 0.09 wt % to about 0.2 wt % of sodium dodecylbenzene sulfonate. According to any one of the above embodiments, the surfactant is safe for use in humans, and in particular for use in cosmetics e.g., such as for use in hair care. According to any one of the above embodiments, the surfactant is a pharmaceutical or cosmetic grade surfactant.

According to one embodiment, the formulation comprises a combination of polyoxyethers of lauryl alcohol, e.g. Laureth-16, Laureth-20, Laureth-21, Laureth-25, Laureth-30, Laureth-38, or Laureth-40 and linear alkyl benzene sulfonate. According to one embodiment, the formulation comprises Laureth-40 and sodium dodecylbenzene sulfonate. According to one embodiment, the formulation comprises from about 3 wt % to about 8 wt % Laureth-40 and from about 0.01 wt % to about 1 wt % sodium dodecylbenzene sulfonate.

According to one embodiment, the formulation comprises from about 3 wt % to about 8 wt % of Sabowax SE40 and from about 0.01 wt % to about 1 wt % sodium dodecylbenzene sulfonate. According to one embodiment, the formulation comprises from about 3 wt % to about 9 wt % of Sabowax SE40 and from about 0.01 wt % to about 2 wt % sodium dodecylbenzene sulfonate.

According to any one of the above embodiments, the amount of solids in said formulation is from about 10 to 50 wt % or from 15 wt % to about 55 wt %. According to one embodiment, the amount of solids is from about 20 to about 45 wt %. According to another embodiment, the amount of solids is from about 20 to about 40 wt %. According to yet another embodiment, the amount of solids in said formulation is from about 22 wt % to about 38 wt % or from about 24 wt % to about 36 wt %.

According to any one of the above embodiments, the glass transition temperature (Tg) of the polymer is from about 10° C. to about 60° C. According to some embodiments, the Tg is from about 10° C. to about 50° C. or to about 40° C. According to yet another embodiment, the Tg is from about 15° C. to about 35° C.

According to any one of the above embodiments, the polymers undergo crosslinking upon drying. The crosslinking occurs via the cross-linking unit of the polymer. According to one embodiment, the crosslinking is a complete crosslinking. According to another embodiment, the crosslinking is a partial crosslinking. Partial crosslinking means that only a part of cross-linkers reacted to form a bond with another polymer. According to some embodiments, partial crosslinking encompasses that about 10 to 90% of the cross-linkers reacted. According to another embodiment, partial crosslinking encompasses about 20% to about 80%, about 30% to about 70%, or about 40% to about 60% of the cross-linkers bound to another polymer. According to some embodiments, upon crosslinking the polymers form a film around hair.

According to any one of the above embodiments, the straightening formulation further comprises a preservative. According to one embodiment, the preservative is selected from propylene glycol, methyl propanediol, caprylyl glycol, polyquaternium −80, didecyldimium chloride, ethylhexylglycerin, Dehydroacetic acid (2-Acetyl-5-hydroxy-3-oxo-4-hexenoic acid δ-lactone), paraben, methyl paraben, phenoxyethanol, iodopropynyl butyl carbamate, and diazolidinyl urea.

According to any one of the above embodiments, the pH of the composition is from 4.5 to 8, from 5 to 7.5, from 5 to 6, from 5.5 to 6.5 or about 5.5.

According to some embodiments, the present invention provides a hair styling formulation from about 1 wt % to about 20 wt % of at least one acrylic-based polymer comprising acrylic units that act as cross-linkers that will form bonds between the polymer chains and from about 0.05 wt % to about 10 wt % of a non-ionic surfactant, wherein the amount of said cross-linker units is from about 1% to about 25 wt % of said acrylic-based polymer, from 0.01 to 3 wt % of an organic compound comprising one or more thiol group and having a molecular weight of from 50 to 1000 Da, and a cosmetically acceptable carrier, wherein the formulation undergoes cross linking and thereby maintain the styling of the hair upon drying. According to some embodiments, the formulation is a hair straightening formulation and the polymers upon drying maintain the hair in straightened conformation. According to such embodiments, the cross-linker is AAEM. According to some such embodiments, the polymer is derived from a combination of acrylic monomers and acrylic oligomers, the combination is selected from the group consisting of (i) monoacrylate oligomer and isobornyl acrylate; (ii) monoacrylate oligomer, isobornyl acrylate and butyl acrylate; and (iii) monoacrylate oligomer, isobornyl acrylate and lauryl methacrylate. According to some such embodiments, the polymers undergo crosslinking upon drying. According to such embodiments, the non-ionic surfactant is selected from Laureth-20, Laureth-21, Laureth-25, Laureth-30, Laureth-38, Laureth-40, Disponil 4060 AFX, and Sabowax SE40. According to some embodiments, the hair straightening formulation comprising from about 2 wt % to about 10 wt % of at least one acrylic-based polymer comprising an acrylic-based cross-linker and from about 0.5 wt % to about 1.5 wt % of a non-ionic surfactant, and from 0.1 to 0.6 wt % of N-acetyl cysteine, wherein the amount of said cross-linker is from about 2.5 wt % to about 25 wt % of said acrylic-based polymer and the non-ionic surfactant is selected from a pharmaceutical or cosmetic grade surfactant and the polymers undergo crosslinking upon drying and the formulation straightens the hair upon drying. According to some embodiments, such formulation further comprises sodium dodecylbenzene sulphonate. According to one embodiment, the formulation comprises from about 0.5 wt % to about 1.5 wt % Laureth having from 20 to 40 units of ethylene oxide and from about 0.05 wt % to about 0.3 wt % sodium dodecylbenzene sulfonate. According to another embodiment, the formulation comprises from about 0.5 wt % to about 1.5 wt % of Sabowax SE40 and from about 0.05 wt % to about 0.3 wt % sodium dodecylbenzene sulfonate. According to such embodiments, the polymer comprises from 85 to 100% of acrylic moieties or consists of acrylic moieties. According to some embodiments, the pH of the formulation is from 5 to 6.

According to some embodiments, the present invention provides a hair straightening formulation comprising from about 3 wt % to about 8 wt % of at least one acrylic-based polymer comprising an acrylic-based cross-linker and from about 0.5 wt % to about 1.5 wt % of a non-ionic surfactant, and from 0.1 to 0.6 wt % of N-acetyl cysteine, wherein the amount of said cross-linker is from about 10 wt % to about 20 wt % of said acrylic-based polymer and the non-ionic surfactant is selected from a pharmaceutical or cosmetic grade surfactant, and wherein the formulation straightens the hair or preserves hair in straightened conformation upon drying. According to such embodiments, the cross-linker is AAEM and the polymer is derived from a combination of acrylic monomers and acrylic oligomers, the combination is selected from the group consisting of (i) a combination of monoacrylate oligomer and isobornyl acrylate; (ii) a combination monoacrylate oligomer, isobornyl acrylate and butyl acrylate; and (iii) a combination monoacrylate oligomer, isobornyl acrylate and lauryl methacrylate, and the non-ionic surfactant is Laureth having from 20 to 40 units of ethylene oxide such as Laureth-20, Laureth-21, Laureth-25, Laureth-30, Laureth-38, Laureth-40, Disponil AFX 4060, and Sabowax SE40. According to some such embodiments, the polymers undergo crosslinking upon drying. According to one embodiment, the monoacrylate oligomer is 2-hydroxy-3-(prop-2-enoyloxy)propyl 2-methyl-2-propylhexanoate. According to some embodiments, such a formulation further comprises from about 0.01 wt % to about 1 wt % of sodium dodecylbenzene sulphonate. According to such embodiments, the polymer comprises from 95 to 100% of acrylic moieties or consists of acrylic moieties. According to some embodiments, the pH of the formulation is from 5 to 6.

According to one embodiment, the hair straightening formulation comprises (i) from 3 to 8 wt % of an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, and AAEM in the amount of from 8 to about 20 wt % of the polymer; (ii) from about 0.3 to about 2 wt % of Laureth-40, Disponil AFX 4060, or Sabowax SE40, (iii) from 0.1 to about 1 wt % of sodium dodecylbenzene sulphonate, and (iv) from 0.1 to 0.6 wt % of NAC. According to another embodiment, the polymer further comprises lauryl methacrylate units in the amount of from about 1 to about 5 wt % of the polymer. According to some embodiments, the pH of the formulation is from 5 to 6. According to one embodiment, the hair straightening formulation comprises from 1 to 7 wt % of the acrylic based polymer.

According to one embodiment, the hair straightening formulation comprises (i) from 4 to 6 wt % of an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, and AAEM in the amount of from 8 to about 20 wt % of the polymer; (ii) from about 0.6 to about 1.5 wt % of Laureth-40, Disponil AFX 4060, or Sabowax SE40, (iii) from 0.1 to about 0.2 wt % of sodium dodecylbenzene sulphonate, and (iv) from 0.2 to 0.5 wt % of NAC. According to another embodiment, the polymer further comprises lauryl methacrylate units in the amount of from about 1 to about 5 wt % of the polymer. According to some embodiments, the pH of the formulation is from 5 to 6.

The term comprising has also the meaning of consisting or consisting essentially of.

According to any one of the above embodiments, the obtained film acts as a surface barrier and/or moisture shield around the hair.

According to some embodiments, the content of the stock emulsion used for the preparation of the ready-to-use formulation of the present invention is as described in Examples 1-2. Each stock emulsion in these tables represents a separate embodiment. According to some particular embodiments, the ready-to-use formulation comprises from 10 to 30 wt % of the stock emulsion and from 0.1 to 0.6 wt % of NAC. According to some particular embodiments, the ready-to-use formulation comprises from 10 to 30 wt % of the stock emulsion EF-117 as defined in Example 2 and from 0.1 to 0.6 wt % or from 0.2 to 0.4 wt % or about 0.3 wt % of NAC. According to some particular embodiments, the ready-to-use formulation comprises from 15 to 25 wt % or about 20 wt % of the stock emulsion EF-117 as defined in Example 2 and from 0.1 to 0.6 wt % or from 0.2 to 0.4 wt % or about 0.3 wt % of NAC. According to some embodiments, the ready-to-use formulation comprises from 12 to 28 wt %, from 14 to 26 wt % or from 15 to 25 wt % of said stock emulsion. According to some embodiments, the ready-to-use formulations are as defined in Tables 3-6.

According to some embodiments, the polymer of the present invention is devoid of maleic anhydride moieties. According to some embodiments, the polymer of the present invention is devoid of maleimide moieties. According to one embodiment, the polymer comprises less than 5 wt %, less than 3 wt % or less than 1 wt % of said moieties. The terms “substantially devoid”, “essentially devoid”, “devoid”, “does not include” and “does not comprise” may be used interchangeably and refer to a formulation that does not include, contain or comprise a particular component, e.g., said formulation comprises less than 0.1 wt %, less than 0.01 wt %, or less than 0.001 wt % of the component. In some embodiments, the term devoid contemplates formulation comprising traces of the devoid component such as traces of a component used in purification process.

According to some embodiments, the ready-to-use formulation of the present invention is a hair styling formulation.

According to any one of the above embodiments, the length of the straightened hair upon drying the formulation is up to 200%, up to 180%, up to 160%, or up to 150%, of the hair's length in un-straightened conformation. According to one embodiment, the length of the straightened hair upon drying the formulation is up to 140%, of the hair's length in un-straightened conformation. According to another embodiment, the length of the straightened hair upon drying the formulation is from 101% to 130%, 105% to 125%, or from 110% to 120% of the hair's length in un-straightened conformation. The un-straightened conformation means the conformation of dry and untreated hair.

According to some embodiments, the formulation of the present invention preserves or maintains hair in the straightened conformation from several hours to several days. According to one embodiment, the formulation of the present invention preserves or maintains hair in the straightened conformation from 1 to 24, from 3 to 20 from 4 to 16 from 6 to 12 hours, provided that that hair is not wetted, washed or shampooed. According to one embodiment, the formulation of the present invention preserves hair in the straightened conformation for from 1 to 5, from 2 to 4, or for 3 days provided that that hair is not wetted, washed or shampooed. According to other embodiments, the formulation of the present invention preserves hair in the straightened conformation for 3 to 14, for 5 to 12 or for 7 to 10 days.

According to some embodiments, the formulation of the present invention preserves hair in the straightened conformation under high humidity conditions. According to one embodiment, the formulation of the present invention preserves hair in the straightened conformation for 1, 2, 4 or 5 days in relative humidity (RH) of more than 50%, 60%, 70% or 80%. The term “relative humidity” as used herein refers to the amount of water vapor present in air expressed as a percentage of the amount needed for saturation at the same temperature (i.e., relative to the “dew point”).

According to any one of the above embodiments, the hair care formulation, e.g., the hair straightening formulation of the present invention is selected from the group consisting of a spray, lotion, cream, foam, mousse, and gel.

According to any one of the above embodiments, the hair care formulation, e.g., the hair straightening formulation of the present invention further comprises one of more additional ingredients or excipients. According to one embodiment, the ingredients are cosmetically acceptable ingredients. According to some embodiments, the additional ingredients are selected from the group consisting of preservatives, thickeners, viscosity modifiers, fatty alcohols, pH adjusting agents, buffers, coloring agents, guar gums, natural gums, perfumes, PEGs, essential oils, silicone fluid dimethicone, hydroxypropyl cellulose, carboxymethyl cellulose and any combination thereof.

According to any one of the above embodiments, the formulation of the present invention has pH of from 5 to 9. According to some embodiments, the pH of the formulation is from 5 to 6.

The pH adjusting agents can be selected from, but are not limited to, glyoxylic acid, maleic acids, succinic acid, formic acid and other organic and inorganic acids.

According to one embodiment, the additional ingredient is a preservative or a combination of preservatives such as propylene glycol, paraben, methyl paraben, phenoxyethanol, caprylyl glycol, iodopropynyl butyl carbamate, and diazolidinyl urea.

According to one embodiment, the essential oil is selected from coconut, lime and argan oil. According to another embodiment, the essential oil is selected from lavender, rosemary, chamomile, cedar-wood, clary sage, jojoba and peppermint oil.

The formulations described hereinabove can also contain a variety of other optional cosmetically acceptable components suitable for rendering such formulations more acceptable. Such conventional optional ingredients are well known to those skilled in the art, e.g., pearlescent aids such as ethylene glycol distearate; preservatives such as benzyl alcohol, methyl paraben, propyl paraben and imidazolidinyl urea; thickeners and viscosity modifiers such as a diethanolamide of a long chain fatty acid (e.g., PEG 3 lauric diethanolamide), cocomonoethanol amide, dimethicone copolyols, guar gum, methyl cellulose starches and starch derivatives, fatty alcohols such as stearyl alcohol, sodium chloride, sodium sulfate, polyvinyl alcohol, and ethyl alcohol; pH adjusting agents such as citric acid, sodium citrate, succinic acid, phosphoric acid, sodium hydroxide, sodium carbonate, etc.; propellants; surfactants; coloring agents such as any of the FD&C or D&C dyes hair oxidizing (bleaching) agents such as hydrogen peroxide, perborate and persulfate salts, hair reducing agents such as the thioglycolates; perfumes; and, sequestering agents such as disodium ethylenediamine tetraacetate, polymer plasticizing agents such as glycerin and propylene glycol. According to some embodiments, said agents generally used individually or collectively at a level of from about 0.01 wt % to about 20 wt %, or from about 0.05 wt % to about 18 wt %, from about 1 wt % to about 10 wt %, from about 2 wt % to about 8 wt % or from about 3 wt % to about 5.0 wt %.

According to one embodiment, the hair straightening formulation comprises at least one of the excipients selected from Sharomix preservative, BEG (ethyl hexyl glycerin, benzyl alcohol, d-alpha—tocopherol 10:89.8:0,2), CP10 (phenoxyethanol: chlorophenesin 90:10), Gum guar, perfume, PEG-12 dimethicone, Silicone fluid Dimethicone (Q7-9120 or 12500 CT), Polydimethylsiloxane (PDMS—350 CST), Hydroxypropyl cellulose (Klucel), Carboxymethylcellulose sodium, and Argan oil, as described in Table 2.

According to any one of the above embodiments, the formulation comprises from about 5 wt % to about 99 wt % of a carrier. According to some embodiment, the formulation comprises from 50 to 98 wt % of from 70 to 95 wt % carrier. According to one embodiment, the carrier is water.

According to yet another aspect, the present invention provides a method of hair styling comprising applying the hair styling formulation of the present invention to the hair and drying the formulation. According to one embodiment, the hair styling is hair straightening. Thus, in one embodiment, the present invention provides a method of hair straightening comprising applying the hair straightening ready-to-use formulation of the present invention to the hair and drying the hair. According to yet another embodiment, the present invention provides a method of preserving hair in a straightened conformation, e.g., after wetting the hair, the method comprises applying hair straightening formulation of the present invention to the hair and drying the formulation, respectively. As used herein, the term “applying” a formulation to the hair or “treating” the hair with a formulation is intended to mean contacting the hair with at least one of the formulations of the invention, in any manner.

According to one embodiment, the drying is drying by heat, e.g., by a fan. According to another embodiment, the drying is by drying at room temperature.

According to any one of the above embodiments, the method comprises combing or brushing the hair to straighten it before drying.

According to any one of the above embodiments, the method comprises leaving the formulation for about 1 to about 10 min on hair before drying. According to some embodiments, the formulation is left for about 2 to about 8 of for about 3 to about 6 minutes.

According to any one of the above embodiments, applying the formulation comprises applying the formulation on wet hair. According to another embodiment, applying of the formulation is applying to dry hair.

As it has been demonstrated in Examples, applying the composition for a plurality of times improves the effect. According to some embodiments, the method comprises applying the formulation a plurality of times. According to some embodiments, the method comprises applying the formulation 2 or 3 times. According to other embodiments, the method comprises applying the formulation 4, 5 or 6 times. According to other embodiments, the method comprises applying the formulation 2, 3, 4, 5 or 6 times/a week.

According to another aspect, the present invention provides a stock emulsion comprising from about 10 wt % to about 50 wt % of at least one acrylic-based polymer comprising acrylic units that act as cross-linkers that will form bonds between the polymer chains and from about 0.5 wt % to about 15 wt % of a non-ionic surfactant, wherein the amount of said cross-linker units is from about 1% to about 25 wt % of said acrylic-based polymer, from 0.1 to 6 wt % of an organic compound comprising one or more thiol group and having molecular weight of from 50 to 1000 Da, a cosmetically acceptable carrier and wherein the composition undergoes cross linking and thereby straightens the hair upon drying. According to some embodiments, the stock emulsion comprises from about 20 wt % to about 40 wt % of at least one acrylic-based polymer. According to other embodiments, the stock emulsion further comprises additional surfactants, such as sodium dodecyl sulfate or alkyl benzene sulfonates. According to some embodiments, the stock emulsion comprises 0.5 to 4 wt % or from 1 to 3 wt % of the organic compound comprising one or more thiol group. According to some embodiments, the organic compound comprising one or more thiol group is selected from a thiol comprising amino acid or a derivative thereof. According to some embodiments, the organic compound comprising one or more thiol group is cysteine. According to another embodiment, the organic compound comprising one or more thiol group is a derivative of cysteine. According to one embodiment, the organic compound comprising one or more thiol group is selected from N-acetyl cysteine, cysteine methyl ester (mecysteine), acetylcysteine, carbocysteine, fudosteine and erdosteine. According to one embodiment, the organic compound comprising one or more thiol groups is N-acetyl cysteine. Thus, according to some embodiments, the present invention provides a stock emulsion comprising from about 10 wt % to about 50 wt % of at least one acrylic-based polymer comprising acrylic units that act as cross-linkers that will form bonds between the polymer chains and from about 0.5 wt % to about 15 wt % of a non-ionic surfactant, wherein the amount of said cross-linker units is from about 1% to about 25 wt % of said acrylic-based polymer, from 0.1 to 6 wt % of NAC. According to some embodiments, the hair straightening ready-to-use formulation is prepared by dissolving from 10 to 40% of from 15 to 25 wt % of the stock emulsion in DDW.

According to another aspect, the present invention provides a hair straightening ready-to-use formulation comprising from 10 to 40 wt % of the stock emulsion of the present invention and a cosmetically acceptable carrier. According to some embodiments, the ready-to-use formulation comprises from 15 to 35 wt % or from 15 to 25 wt % of the stock emulsion of the present invention and a cosmetically acceptable carrier.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the formulation or component may include additional ingredients, but only if the additional ingredients do not materially alter the basic and novel characteristics of the claimed formulations or methods.

As used herein, the term “about”, when referring to a measurable value such as an amount, a temporal duration, and the like, is meant to encompass variations of +/−10%, or +/−5%, +/−1%, or even +/−0.1% from the specified value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The term “or,” as used herein, denotes alternatives that may, where appropriate, be combined; that is, the term “or” includes each listed alternative separately as well as their combination if the combination is not mutually exclusive.

Having now generally described the invention, the same will be more readily understood through reference to the following examples, which are provided by way of illustration and are not intended to be limiting of the present invention.

### Examples

The following non-limiting examples describe the synthesis of self-cross-linking acrylic stock emulsions and their incorporation into personal care, ready-for-use hair styling, and specifically, hair-straightening formulations.

### Example 1: Synthesis of Stock Emulsions Comprising Acrylic Polymers with Crosslinking Units

A general procedure for the preparation of the acrylic copolymers comprising crosslinking units is described.

### Pre-Emulsion Preparation:

37.5 g sodium dodecylbenzene sulfonate was completely dissolved in 242 g deionized water by mixing with a magnetic stirrer. Then 27 g Laureth-40 (60% in deionized water) were added to form a transparent viscous surfactant solution. In a 0.5 liter beaker were placed all of the monomers (see below). This monomer solution was poured into the surfactant solution and mixed to give a pre-emulsion. The pre-emulsion was kept for 20 hours in a refrigerator.

### Monomers/Oligomers:

60 g CN152 (Sartomer), Epoxy acrylate oligomer
67.5 g isobornyl monoacrylate,
22.5 g 2-(methacryloyloxy)ethylacetoacetate (AAEM)—the cross-linker,
Water, 342 g.

CN152 of Sartomer comprises 2-hydroxy-3-(prop-2-enoyloxy)propyl 2-methyl-2-propylhexanoate as a main component. It may contain traces of the initial ingredients such as 2,3-Epoxypropyl neodecanoate, neodecanoic acid 1-ester, 1,2,3-propanetriol mono-2-propenoate, acrylic acid and mequinol.

### Polymerization

To the pre-emulsion 300 mg ammonium persulfate were added and it was stirred with a magnetic stirrer for 0.5 h. 100 ml of water were added to the reactor and the mechanical stirrer was set at 300 rpm. Then 100 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 30 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 2.7. The pH was raised to 6.8 by carefully adding about twenty drops of 25% ammonium hydroxide. This was monitored using a pH electrode. The final yield was 500 ml.

### Analyses

The resulting polymer emulsion (stock emulsion) was analyzed for its glass transition temperature, Tg, and the solid content. The Tg by Differential Scanning Calorimeter (DSC) was 17° C., and the total solids content was 32.8%.

Several additional polymers with different content of the monomers/oligomers were prepared as described above. Table 1 summarizes the initial ingredients and properties of the obtained products.

TABLE 1
Different preparations of stock emulsions.

| | | | batch | | batch | | batch | | batch | | batch | |
|---|---|---|---|---|---|---|---|---|---|---|---|---|
| | 1140 | | 2-18-2 | | 2-18-7 | | 2-18-15 | | 2-18-19 | | 2-18-24 | |
| Materials | wt, g | wt % | wt, g | wt % | wt, g | wt % | wt, g | wt % | wt, g | wt % | wt, g | wt % |
| Monoacrylate | 60 | 11.54 | 60 | 9.63 | 60 | 9.81 | 60 | 11.47 | 60 | 11.47 | 60 | 11.4 |
| Oligomer CN | | | | | | | | | | | | |
| 152 | | | | | | | | | | | | |
| Isobornyl | 67.5 | 12.9 | 67.5 | 10.8 | 67.5 | 11.0 | 54 | 10.3 | 54 | 10.32 | 67.5 | 12.8 |
| Acrylate (Sigma) | | | | | | | | | | | | |
| Butyl Acrylate | | | | | | | 13.5 | 2.58 | — | | — | — |
| Lauryl | | | | | | | | | 13.5 | 2.58 | — | — |
| Methacrylate | | | | | | | | | | | | |
| AAEM (cross- | 22.5 | 4.30 | 22.5 | 3.61 | 11 | 1.80 | 22.5 | 4.3 | 22.5 | 4.30 | 27 | 5.1 |
| linker) | | | | | | | | | | | | |
| Laureth-40 (non- | 27 | 5.16 | 27 | 4.33 | 27 | 4.41 | 27 | 5.2 | 27 | 5.16 | 27 | 5.1 |
| ionic surfactant) | | | | | | | | | | | | |
| Sodium | 3.75 | 0.72 | 3.75 | 0.60 | 3.75 | 0.61 | 3.75 | 0.72 | 3.75 | 0.72 | 3.75 | 0.71 |
| Dodecylbenzene | | | | | | | | | | | | |
| sulphonate | | | | | | | | | | | | |
| (surfactant) | | | | | | | | | | | | |
| Ammonium | 0.33 | 0.06 | 0.33 | 0.05 | 0.33 | 0.05 | 0.33 | 0.06 | 0.33 | 0.06 | 0.33 | 0.06 |
| persulphate | | | | | | | | | | | | |
| DIW | 342 | 65.48 | 442 | 70.9 | 442 | 72.27 | 342 | 65.38 | 317 | 65.38 | 342 | 64.82 |
| Total | 523. | 100. | 623.0 | 100.0 | 611.5 | 100.0 | 523.0 | 100.0 | 527.5 | 100.0 | 527.5 | 100.0 |
| Percent of solids | 32.5 | | | 27.3 | | 26.0 | | 32.5 | | 34.1 | | 33.1 |
| calculated,% | | | | | | | | | | | | |
| % solids by | | | | 26.7 | | | | 34.6 | | — | | — |
| lyophilization | | | | | | | | | | | | |
| Percent solids by | | | | 26.6 | | 26.1 | | 34.9 | | 35.4 | | 33.8 |
| DSC,% | | | | | | | | | | | | |
| Tg ° C. by DSC | 17 | | | | | 44.1 | | 38.4 | | NA | | N.A. |
| Ammonium | 12 | | 12 | | 12 | | 10 | | 12 | | 18 | |
| Hydroxide, 25% | | | | | | | | | | | | |
| (drops) | | | | | | | | | | | | |

### Example 2. Preparation of Stock Emulsions EF-101, EF-105, EF-117 and EF-120

### Stock Emulsion EF-101

### Pre-Emulsion Preparation:

7.5 g sodium dodecylbenzene sulfonate was completely dissolved in 500 ml deionized water by mixing with a magnetic stirrer. Then 46.6 g Disponil AFX 4060 (Cognis) (fatty acid alcohol polyglycol ether) were added to form a transparent viscous surfactant solution. In a 1 liter beaker were placed all of the monomers (see below). This monomer solution was poured into the surfactant solution and mixed to give a pre-emulsion. The pre-emulsion was kept for 20 hours in a refrigerator.

### Monomers/Oligomers:

110 g CN152 (Sartomer), (2-hydroxy-3-(prop-2-enoyloxy)propyl 2-methyl-2-propylhexanoate)
129 g isobornyl monoacrylate,
41.5 g 2-(methacryloyloxy)ethylacetoacetate (AAEM)—the cross-linker,
Water, 700 g.

### Polymerization

To the pre-emulsion 580 mg ammonium persulfate were added and it was stirred with a mechanical stirrer for 0.5 h. 200 ml of water were added to the reactor and the mechanical stirrer was set at 300 rpm. Then 200 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 58 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 2.7. The pH was raised to 6.8 by carefully adding about twenty drops of 25% ammonium hydroxide. This was monitored using a pH electrode. The final yield was 980 ml. The total solids content was 33%. The acrylic copolymer was 19.6% of the emulsion.

### Stock Emulsion EF-105

### Pre-Emulsion Preparation:

3.75 g sodium dodecylbenzene sulfonate was completely dissolved in 250 g deionized water by mixing with a magnetic stirrer. Then 23.3 g Sabowax SE40 (polyethylene glycol-40 stearate) cosmetic grade surfactant, were added to form a transparent viscous surfactant solution. In a 1 liter beaker were placed all of the monomers (see below). This monomer solution was poured into the surfactant solution and mixed to give a pre-emulsion. The pre-emulsion was kept for 20 hours in a refrigerator.

### Monomers/Oligomers:

55 g CN152 (Sartomer), Epoxy acrylate oligomer
64.5 g isobornyl monoacrylate,
20.7 g 2-(methacryloyloxy)ethylacetoacetate (AAEM)—the cross-linker,
Water, 350 g.

### Polymerization

To the pre-emulsion 290 mg ammonium persulfate were added and it was stirred with a mechanical stirrer for 0.5 h. 100 mls of water were added to the reactor and the mechanical stirrer was set at 300 rpm. Then 100 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 29 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 2.7. The pH was raised to 6.8 by carefully adding about twenty drops of 25% ammonium hydroxide. This was monitored using a pH electrode. The final yield was 440 ml and the total solids content was 32%. The acrylic copolymer was 18.3% of the emulsion.

### The Stock Emulsion EF-120

The stock emulsion EF-120 was prepared as described below.

### Materials:

Acrylic Monomers
Isobornyl Acrylate (IBA)=31.7 g
CN152=27 g
Eastman™ AAEM Cross-linker=10.2 g
Total Monomers=Total Polymer=68.9 g

### Surfactants:

Sodium dodecylbenzene sulfonate, ˜80%=1.85 g
Sabowax SE 40 (PEG −40 stearate)=11.41 g
Total Surfactants (as solids)=13.26 g

### Initiator:

Ammonium persulfate (APS)=0.15 g

### Others:

Deionized Water 100 g

### Pre-Emulsion Preparation:

Sabowax SE 40-11.41 g and sodium dodecylbenzene sulfonate-1.85 g were stirred in 60 ml of deionized water at room temperature until a clear solution was formed. In a separate vessel, the monomers CN 152-27 g, IBA 31.7 g and AAEM 10.2 g were mixed and stirred with a magnetic stirrer until the solution became clear. The solution was viscous. Then the solution of acrylate monomers was added to the solution of surfactants with magnetic stirring to form a white pre-emulsion. This pre-emulsion was left overnight in refrigerator at 4° C.

### Polymerization:

40 ml of water were added to the reactor and the mechanical stirrer was set at 300 rpm. 100 mls of pre-emulsion were added with 0.075 g of APS. Nitrogen gas purged through the mixture for 30 minutes. The reactor was then placed in an oil bath pre-heated to 92° C. The temperature in the reactor rapidly reached 78° C. and a weak (below condenser) reflux was observed. The temperature continued to rise, but slowly, and after two hours it reached 83.5° C. and then stabilized at 83-84° C. The remainder of the pre-emulsion was added to the reactor over two hours using a peristaltic pump. The reaction temperature in the final stages of monomer addition remained around 85° C. When all of the monomers were inside the reactor, an additional 0.075 g of ammonium persulfate were added and the temperature was raised to 95-97° C. for an additional hour to complete the polymerization. Then the contents of the reactor were cooled slowly with stirring overnight. The emulsion polymer product, a synthetic latex, was removed from the reactor and filtered through a synthetic non-woven material (kitchen clean pad). No coagulation product was observed. The pH of the emulsion was below 3.4. The emulsion obtained was stable and showed no signs of separation during storage. The resulted stock emulsion comprises 37.9% (68.9/182) by weight acrylic based polymers. The calculated content of all solids in the stock emulsion is 45.1% by weight. The percent of solids in the resulted stock emulation as calculated by DSC and LOD was 43% and 45.7%, respectively.

### The Stock Emulsion EF-117

A general procedure for preparation of the acrylic copolymers comprising crosslinking units is described.

### Pre-Emulsion Preparation:

22.5 g sodium dodecylbenzene sulfonate was completely dissolved in 1.5 L deionized water by mixing with a magnetic stirrer. Then 140 g Sabowax SE40 were added to form a transparent viscous surfactant solution.

The Following Monomers were Mixed in a Beaker:
Epoxy Acrylate Oligomer, CN 152 (Sartomer)=330 g
Isobornyl Acrylate (Sigma)=387 g
(Methacryloyloxy)ethylacetoacetate AAEM (Sigma)=124.4 g.

This monomer solution was poured into the surfactant solution and mixed to give a pre-emulsion. The pre-emulsion was kept for 20 hours in a refrigerator.

CN152 of Sartomer comprises 2-hydroxy-3-(prop-2-enoyloxy)propyl 2-methyl-2-propylhexanoate as a main component. It may contain traces of the initial ingredients such as 2,3-Epoxypropyl neodecanoate, neodecanoic acid 1-ester, 1,2,3-propanetriol mono-2-propenoate, acrylic acid and mequinol.

### Polymerization

To the pre-emulsion 1.74 g ammonium persulfate were added and it was stirred with a mechanical stirrer for 0.5 h. 600 ml of water were added to the reactor and the mechanical stirrer was set at 300 rpm. Then 600 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 174 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 3.79. Polymer concentration between 30% to 50% in aqueous solution. The amount of the solids is about 33.3% as measured by DSC to about 39.5 as measured by NOD.

### Example 3: Preparation of Ready-for-Use Hair Straightening Formulations

Table 2 lists chemicals and auxiliary materials that may be used in the hair straightening formulation of the present application.

TABLE 2

| | | Trade | |
|---|---|---|---|
| | | Name/ | |
| | Ingredient | Supplier | CAS |
| 1 | Double Deionized Water | Zalion | |
| 2 | Sharomix Preservative | Sharon Labs | BEG: |
| | BEG (ethyl hexyl glycerin, | | 70445-33-9; |
| | benzyl alcohol, d- | | 100-51-6; |
| | alpha-tocopherol | | 59-02-9 |
| | 10:89.8:0.2) or CP10 | | CP-10: |
| | (phenoxyethanol: | | 122-99-6; |
| | chlorophenesin 90:10) | | 104-29-0 |
| 3 | Gum guar | Solvay | |
| 4 | Perfume | | |
| 5 | PEG-12 dimethicone/ | Dow Corning | |
| | 193 C Fluid | | |
| 6 | Silicone fluid Dimethicone | Dow corning | 63148-62-9 |
| | Q7-9120 | | |
| | 12500 CT or 350CST- | | |
| | Polydimethylsiloxane | | |
| | (PDMS) | | |
| 7 | Hydroxypropyl cellulose | Ashland | EF Ref—420705 |
| | Klucel | | HF Ref—424941 |
| 8 | Carboxymethylcellulose | Spectrum | 9004-32-4 |
| | sodium-CA194 | | |
| 9 | Argan oil/Argania Spinosa | | |

The following ready-to-use hair straightening formulations are prepared by mixing the stock emulsion prepared as described in Examples 1-2 in double distilled water with the addition of N-acetyl cysteine (NAC). The final concentration of the stock emulsion in the ready-to-use formulation is from 10 to 30 wt % and the concentration of NAC in the ready-to-use formulation is from 0.2 to 0.6 wt %, as described in Table 3. The content of the stock emulsion in the ready-to-use formulation is indicated as “% of stock emulsion”.

TABLE 3
Ready-to-use hair straightening formulations.

| | Formulation No. | | | | | | | |
|---|---|---|---|---|---|---|---|---|
| | A | B | C | D | E | F | G | H |
| stock emulsion | 2-18-2 | 2-18-19 | EF-101 | EF-105 | EF-120 | EF-101 | EF-105 | EF-120 |
| Batch | | | | | | | | |
| wt % of stock | 10-30 | 10-30 | 10-30 | 10-30 | 10-30 | 20 | 20 | 20 |
| emulsion | | | | | | | | |
| NAC | 0.2-0.6 | 0.2-0.6 | 0.2-0.6 | 0.2-0.6 | 0.2-06 | 0.3 | 0.3 | 0.3 |
| Water | 69.4-89.8 | 69.4-89.8 | 69.4-89.8 | 69.4-89.8 | 69.4-89.8 | 79.7 | 79.7 | 79.7 |

All formulations are stable and no phase separation was observed after 24 hours.

### Example 4. Preparation of Ready-to-Use Hair Straightening Formulation Based on EF-117

The following ready-to-use hair straightening formulations were prepared by mixing the stock emulsion EF-117, prepared as described in Example 3, in double distilled water with the addition of N-acetyl cysteine (NAC). The final concentration of the stock emulsion in the ready-to-use formulation is from 10 to 30 wt % and the concentration of NAC is from 0.2 to 0.6 wt %; the formulations comprising NAC had. Ready for use formulations are presented in Tables 4 and 5.

TABLE 4
Ready-to-use hair straightening formulations based on EF-117 stock emulsion.

| | Formulation No. | | | | | | | | |
|---|---|---|---|---|---|---|---|---|---|
| | I | J | K | L | M | N | O | P | Q |
| Stock emulsion | EF-117 | EF-117 | EF-117 | EF-117 | EF-117 | EF-117 | EF-117 | EF-117 | EF-117 |
| Batch | | | | | | | | | |
| wt % of stock | 15 | 15 | 15 | 20 | 20 | 20 | 25 | 25 | 25 |
| emulsion | | | | | | | | | |
| NAC (wt %) | 0.3 | 0.4 | 0.5 | 0.3 | 0.4 | 0.5 | 0.3 | 0.4 | 0.5 |
| Water (wt %) | 84.7 | 84.6 | 84.5 | 79.7 | 79.6 | 79.5 | 74.7 | 74.6 | 74.5 |

TABLE 5
Ready-to-use hair straightening
formulations based on EF-117 stock emulsion.

| | Formulation No. | | |
|---|---|---|---|
| | R | S | T |
| Stock emulsion | EF-117 | EF-117 | EF-117 |
| Batch | | | |
| wt % of stock | 20 | 20 | 20 |
| emulsion | | | |
| NAC (wt %) | 0.125 | 0.25 | 0 |
| Water (wt %) | 79.7 | 79.6 | 80 |

### Example 5. Applying Hair-Straightening Formulation to Hair

The styling cream may be applied on both dry and wet hair. Described herein is a general procedure for applying the hair straightening formulation after washing the hair.

After washing the hair rinse out the shampoo using warm water and wring out the hair removing as much water as possible. Pour a small portion of the formulation into the palm of your hand; about 0.15 g for 1 g of hair, length 30 cm. Run the formulation through the length of the hair, trying to apply it to every strand. Allow the formulation to set for about 1 to 5 min. The longer the time the formulation stays on the hair, the more effective it is in straightening and protecting the hair. Use a wide-tooth comb to comb the hair after applying the leave-in formulation. This will spread the product out even further, and help to avoid clumps of the formulation from making certain sections of the hair greasy while others are left dry.

Start air drying the hair with a fan on a medium heat and high speed setting.

### Methods

In all following examples, the experiments were performed on swatches of Brazilian hair type 2 (initial untreated swatches are presented in FIG. 1). The swatches were weighed and their end-to-end lengths were measured. The swatches were wetted, combed, and their length in straightened form was measured; all swatches at their extended state were about 24 cm long. After application of the formulation, the swatches were air-dried and combed 3 times, unless stated otherwise. In case of multiple applications, the swatches were wetted (shampooed and washed) and combed (3-5 times) before each additional application.

### Example 6. Efficacy of Ready-to-Use Formulations Based on EF-117 Stock Emulsion

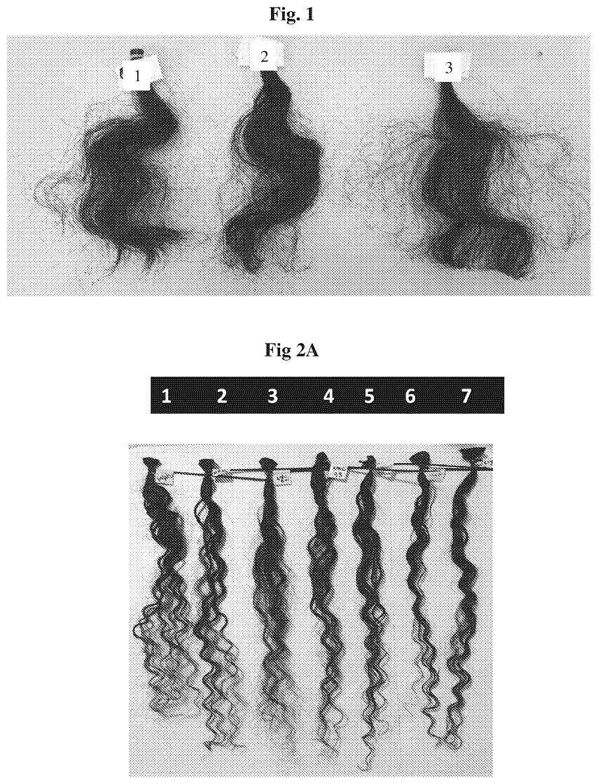

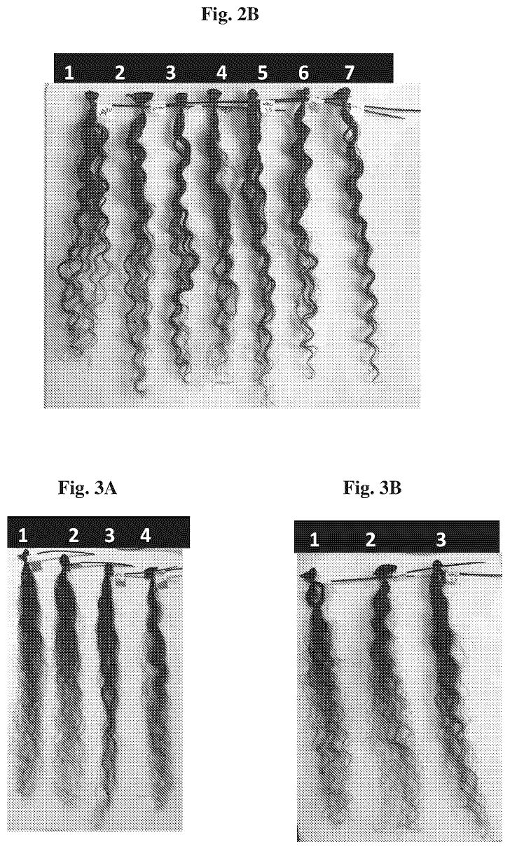

The following formulations were prepared and tested. The experimental arrangement and the results are summarized in Table 6. The results are shown in FIG. 2A-2B. The numbers of the formulations as appear in Table 6 correspond to the numbers in the Figures.

TABLE 6
Tested formulations

| Formulation No. | 1 | 2 | 3 | 4 | 5 | 6 | 7 |
|---|---|---|---|---|---|---|---|
| Stock emulsion Batch | EF-117 | EF-117 | EF-117 | EF-117 | EF-117 | EF-117 | EF-117 |
| wt % of stock emulsion | 0 | 20 | 20 | 20 | 20 | 20 | 20 |
| NAC (wt %) | 0 | 0.0 | 0.5 | 0.4 | 0.3 | 0.25 | 0.125 |
| Water (wt %) | 100 | 80 | 79.5 | 79.6 | 79.7 | 79.75 | 79.875 |

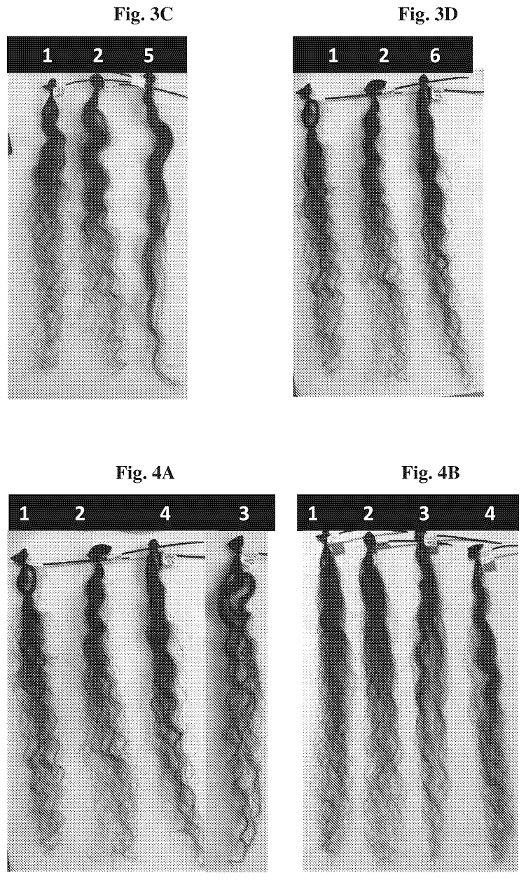

Swatches were treated with formulations 1-7 (swatch 1 was used as a control). Each swatch was treated with the tested formulation, combed and brushed and lightly dried with a domestic hair drier (FIG. 2A). After measuring the maximum swatch lengths, they were placed in a humidity chamber at 20° C. and 80% relative humidity for three days. The results are presented in FIG. 2A-2B showing swatches immediately after treatment (FIG. 2A) and 3 days in humid conditions after the treatment (FIG. 2B).

After three days of exposure to the extreme relative humidity of 80% (see FIG. 2B), swatches treated with the formulation comprising 0.3 wt % of NAC showed the best effect in maintaining an extended and narrow look. It can be seen that all formulations comprising NAC had better look than formulations without NAC.

### Example 7. Influence of Different Parameters on Efficacy of Formulations Based on EF-117 Stock Emulsion

The influence of a number of applications on hair straightening was tested. First, the number of applications was tested. Swatches 1 and 2 were used as a control, the rest swathes contained were treated with formulations comprising 0.3 wt % of NAC, 20 wt % EF −117 in DDW and applied a different number of times according to the schedule described in Table 7.

TABLE 7
Experimental design

| 1 | 2 | 3 | 4 | 5 | 6 |
|---|---|---|---|---|---|
| DDW | EF-117 20% | 2 | 1 | 3 | 4 |
| | in DDW | applications | application | applications | applications |
| Air | 2 applications | Air dry | Air dry | Air dry | Air dry |
| dry | Air dry | | | | |

The applications were performed according to the following schedule:
1^{st} application on 10-Jan.-2021; 2^{nd}—on 18-Jan.-2021; 3^{rd}—on 19-Jan.-2021; and 4^{th}—on 20-Jan.-2021; after the application, the hair was combed 3 times.

The results are presented in FIG. 3A-3D. The swatches were pleasant to the touch. An improvement in hair straightening was observed as the number of treatments increased.

Next, the effect of different concentrations of NAC was tested according to the arrangement described in Table 8. Each swatch was applied 3 times as disclosed in Table 8.

TABLE 8
Experimental design

| 1 | 2 | 3 | 4 | Applications |
|---|---|---|---|---|
| Ref | EF 117 20% | 1% NAC in | 0.3% NAC in | 1^{st} application |
| DDW | in DDW | EF 117 20% | EF 117 20% | 18-Jan.-2021 |
| Air | Air dry | Air dry | Air dry | 2^{nd} application |
| dry | | | | 19-Jan.-2021 |
| | | | | 3^{rd} application |
| | | | | 20-Jan.-2021 |

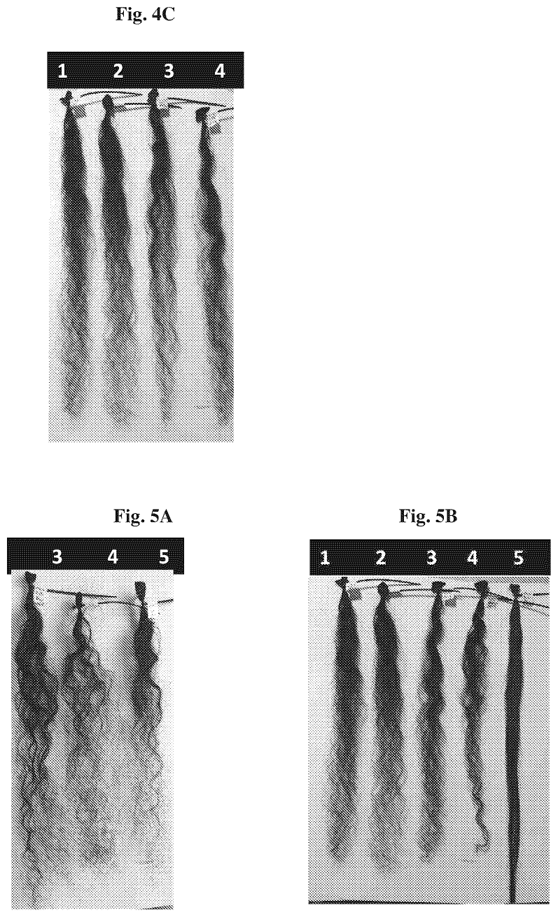

The results are presented in FIG. 4A-4C. After one application, there was no difference between 0.3% and 1% NAC but, after the second treatment, 1% NAC provided slightly better results. After 3 applications, the results obtained with 1% were a little better than 0.3%. However, the application of the formulation comprising 1% NAC resulted in an unpleasant smell. This smell transferred to the hair and dispersed after a few days.

Next, the effect of different drying techniques was tested.

The experiment was performed as described in Table 9. Swatches 3-5 were treated with 0.3 wt % of NAC in EF-117 20% dissolved in DDW

TABLE 9
Experimental design—effect of trying techniques

| | 1 | 2 | 3 | 4 | 5 |
|---|---|---|---|---|---|
| 18-Jan.-2021 | Ref | EF-117 20% | Air dry | Blow | Flat iron |
| | DDW | in DDW | | dryer | dryer 200° C. |
| Washing then | Ref | EF-117 20% | Air dry | Blow | Flat iron |
| treatment | DDW | in DDW | | dryer | dryer 200° C. |
| 19-Jan.-2021 | | | | | |
| Washing then | Ref | EF-117 20% | Air dry | Blow | Flat iron |
| treatment | DDW | in DDW | | dryer | dryer 200° C. |
| 20-Jan.-2021 | | | | | |

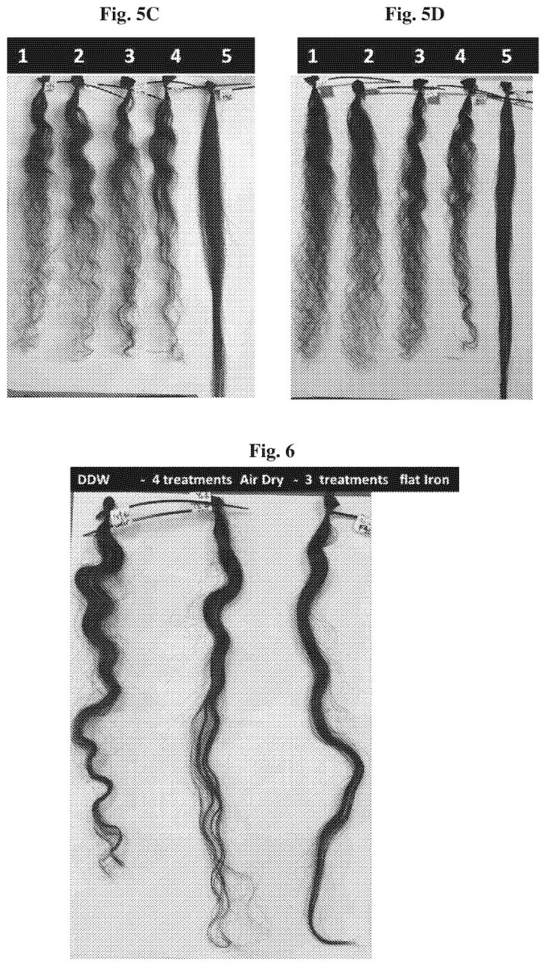

The results are present in FIGS. 5A-5D. It can be seen that after 1 or 2 applications there was no real difference between drying methods. After a few applications, the hair dried with flat Iron at 230° C. showed really better results than hair dried at room or at 60° C.

The effect of air drying or treating with flat iron at 230° C. was additionally tested. The swatches were treated with formulations comprising 20 wt % of EF-117 stock emulsion and 0.3% NAC and then were air-dried (25° C.) or dried with a flat iron at 230° C. The results are presented in FIG. 6. As can be seen, that air drying (25° C.) was equivalent to drying with a flat iron at 230° C. However, drying with flat iron caused smoke and left a sticky layer on the fingers. The strand formed was very rigid and required combing out. Therefore, it was concluded that blow drying is a preferred method of hair drying when using the composition of the present invention.

In additional examples, the composition was applied on dry hair and the results were similar to those when the composition was applied on wet hair. Therefore, it is not important whether the composition is applied on dry or wet hair.

Although the present invention has been described hereinabove by way of preferred embodiments thereof, it can be modified, without departing from the spirit and nature of the subject invention as defined in the appended claims.

1-31. (canceled)

32. A hair styling ready-to-use formulation comprising from about 1 wt % to about 20 wt % of at least one acrylic-based polymer comprising acrylic units that act as cross-linkers that will form bonds between the polymers, wherein the amount of said cross-linker units is from about 2.5% to about 25 wt % of said acrylic-based polymer, from about 0.05 wt % to about 6 wt % of a non-ionic surfactant, from 0.01 to 0.9 wt % of an organic compound comprising one or more thiol group and having a molecular weight of from 50 to 1000 Da, and a cosmetically acceptable aqueous carrier, wherein the polymer chains undergo cross-linking and thereby styling the hair upon drying.

33. The hair styling ready-to-use formulation of claim 32, comprising from 0.1 to 0.8 wt % or from 0.2 to 0.6 wt % of the organic compound comprising one or more thiol groups.

34. The hair styling ready-to-use formulation of claim 32, wherein (i) the organic compound comprising one or more thiol groups has a molecular weight of from 100 to 400 Da, (ii) the organic compound comprising one or more thiol groups is selected from the group consisting of cysteine and derivative thereof, captopril, and glutathione or (iii) both (i) and (ii).

35. The hair styling ready-to-use formulation of claim 32, wherein the organic compound comprising one or more thiol groups is N-acetyl cysteine.

36. The hair styling ready-to-use formulation of claim 32, wherein the formulation is characterized by at least one of:
(i) the pH of the formulation is from 4.5 to 9 or from 5 to 6;
(ii) the polymer is derived from acrylic monomers, acrylic oligomers or a combination thereof;
(iii) the monoacrylate oligomer is 2-hydroxy-3-(prop-2-enoyloxy)propyl 2-methyl-2-propylhexanoate;
(iv) the amount of said cross-linkers is from about 5 to about 20 wt % of said acrylic-based polymer;
(v) said cross-linker units and moieties of acrylic monomer and/or acrylic oligomers collectively constitute from 85 to 100% of the polymer;
(vi) the non-ionic surfactant is selected from the group consisting of ethoxylated and/or propoxylated alcohol or ester, Laureth-20, Laureth-21, Laureth-25, Laureth-30, Laureth-38, Laureth-40, tyloxapol, polysorbate (TWEEN), Disponil AFX 4060, Sabowax SE40, PPG-5-ceteth-20, PEG-20 sorbitan isostearate, alkoxylated alcohols, alkoxylated polyol esters, decyl glucoside, coco-glucoside, sucrose laurate, sorbitol, polyoxyethylene, sorbitan monolaurate and any combination thereof; and
(vii) comprising from about 0.2 wt % to about 4 wt % of said surfactant.

37. The hair styling ready-to-use formulation of claim 36, wherein the formulation is characterized by at least one of:
(i) the acrylic monomers and acrylic oligomers are selected from the group consisting of monoacrylate oligomer, epoxy monoacrylate oligomer, acrylate, methacrylate(s), isobornyl acrylate, butyl acrylate, lauryl methacrylate, 2-hydroxy-3-(prop-2-enoyloxy)propyl 2-methyl-2-propylhexanoate and any combination thereof;
(ii) the cross-linker unit is 2-(methacryloyloxy)ethylacetoacetate (AAEM).

38. The hair styling ready-to-use formulation of claim 38, wherein the polymer is derived from a combination of acrylic monomers and acrylic oligomers, said combination is selected from the group consisting of (i) a combination of a monoacrylate oligomer and isobornyl acrylate; (ii) a combination of a monoacrylate oligomer, isobornyl acrylate and butyl acrylate; and (iii) a combination of a monoacrylate oligomer, isobornyl acrylate and lauryl methacrylate.

39. The hair styling ready-to-use formulation of claim 32, wherein the formulation comprises from about 1.5 wt % to about 12 wt % of the polymer comprising cross-linkers in the amount of about 3 wt % to about 20 wt % of said polymer, from about 0.2 wt % to about 4 wt % of said non-ionic surfactant, and from 0.2 to 0.8 of N-acetyl cysteine.

40. The hair styling ready-to-use formulation of claim 39, wherein the polymer comprises 2-hydroxy-3-(prop-2-enoyloxy)propyl 2-methyl-2-propylhexanoate.

41. The hair styling ready-to-use formulation of claim 39, wherein the formulation comprises from 3 to 7 wt % of the polymer and the pH of the formulation is from 4.5 to 6.5.

42. The hair styling ready-to-use formulation of claim 39, wherein solids of said formulation is from about 10 wt % to about 50 wt %; (ii) the cosmetically acceptable aqueous carrier is water or (iii) both (i) and (ii).

43. The hair styling ready-to-use formulation of claim 32, wherein the hair straightening formulation is selected from the group consisting of a spray, lotion, cream, foams, mousse, and gels.

44. The hair styling ready-to-use formulation of claim 32, further comprising a cosmetically acceptable excipient.

45. The hair styling ready-to-use formulation of claim 44, wherein the cosmetically acceptable excipient is selected from the group consisting of preservatives, thickeners, viscosity modifiers, fatty alcohols, pH adjusting agents, buffers, coloring agents, guar gums, perfumes, PEGs, essential oils silicone fluid dimethicone, hydroxypropyl cellulose, carboxymethyl cellulose and any combination thereof.

46. The hair styling ready-to-use formulation of claim 32, wherein the hair styling comprises hair straightening.

47. The hair styling ready-to-use formulation of claim 46, wherein the formulation straightens the hair upon drying.

48. A method of styling hair comprising applying the formulation of claim 32 to the hair and drying the formulation.

49. The method of claim 48, wherein styling hair comprises hair straightening.

50. The method of claim 48, wherein the hair is selected from type 3 and 4 hair.

51. The method of claim 48, wherein the method is characterized by at least one of:
(i) the method further comprises the step of combing or brushing the hair to straighten it before drying;
(ii) formulation is applied to dry or wet hair;
(iii) the formulation is applied to the hair a plurality of times; and
(iv) the drying is selected from drying by heat or drying at room temperature.
